SEQUENCE LISTING (111 sequences)

SEQ ID NO: 1 - DNA, 265 bp, Artificial sequence
  StoChaSeq
  misc_feature (34)..(226): N is c, g, t or a
  misc_feature (231)..(238): N is c, g, t or a
gaattcgcggccgcttctagagggtctcatgccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggagnnnnnnnngagacctactagtagcggccgctgcag

SEQ ID NO: 2 - DNA, 265 bp, Artificial sequence
  StoChaSeq_RC
  misc_feature (28)..(35): N is c, g, t or a
  misc_feature (40)..(232): N is c, g, t or a
ctgcagcggccgctactagtaggtctcnnnnnnnnctccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggcatgagaccctctagaagcggccgcgaattc

SEQ ID NO: 3 - DNA, 277 bp, Artificial sequence
  23892648-6
cctatttttataggttaatgtcatgataataatggtttcttagacgtcaggtggcacggtctcatgccgccgcttgtactagacttctcgtatctactgtggcaaagcgtgatgcaggatgaggcaatataggtttgtgtggactgcatcggaatgctacggtatagtgtgttggtaccggccttctatgggtggaatacgatctgtcttaagtatagctgataatggaggggtaagagcccacaggcatcgtcggagtcgtatgatgagtattcaa

SEQ ID NO: 4 - DNA, 283 bp, Artificial sequence
  23885241-6
cctatttttataggttaatgtcatgataataatggtttcttagacgtcaggtggcacggtctcatgcccaagaacaaaaaaggcaattattgtcgcgcgccatcagttttaatatatatcagcgtggctcgtaattgatgagtcgtaaagttaggctctgaacgttctgtcggatttgatgactgttggtaaaatccagcttcagatagtgcagagttgtatgctcaactagctatttaattcgtaagtatatcgactaaggagtcgtggcatgagtattcaa

SEQ ID NO: 5 - DNA, 284 bp, Artificial sequence
  23885172-6
cctatttttataggttaatgtcatgataataatggtttcttagacgtcaggtggcacggtctcatgccgacggttttcacattgcttatgtatccattttgtggccaggagttttccgttatcatttttttacgaaatatctcccggtacatgttgtctcgggcttgaaagaaactgaaactgtacacctgtgtggggtggatcttaataggctggatgtggttgtggtctaggaagagtaatcccataaagttttctcgtggagtgtggggatgagtattcaa

SEQ ID NO: 6 - DNA, 284 bp, Artificial sequence
  23883897-6
cctatttttataggttaatgtcatgataataatggtttcttagacgtcaggtggcacggtctcatgccgtgatacctagtatccttataggtagagttggctcttggaaagcatggggcactggcgcgacgtatagggtttcatatacaatccaaataatctgccttgcaggaactcccatctgggaattacctctggtgagaagttcagggaaggaagttgggtcggtggagatggatgtggcaaatggcgtaattaattggaggtccgggatgagtattcaa

SEQ ID NO: 7 - DNA, 284 bp, Artificial sequence
  23884068-6
cctatttttataggttaatgtcatgataataatggtttcttagacgtcaggtggcacggtctcatgccctctccgacctgttctacgcaggtgcatgcatatcttgattttaaataatacatgaaagatcattagtgattttgaatctataagattgcatgcaactacactatcggactacgtagtcattgcggatggtcgtcaatgtacccagatgtacgtcgatctgggttcgcgcacaagcttatatttctgagtccaggagttctgtgatgagtattcaa

SEQ ID NO: 8 - DNA, 280 bp, Artificial sequence
  23883672-6
cctatttttataggttaatgtcatgataataatggtttcttagacgtcaggtggcacggtctcatgccacctcgccaatgcggatgaatgatttacgacattacggacgcttgtaccattgtactaagcttgtgctatcatgattgtcggatgatttctttgctggcgaggggaaccaacaggcggctatagaaggggttgaagaattggcaccagttggttagtcggcaggagtttggaccgcgcgaatcaagcatggaggtggcggatgagtattcaa

SEQ ID NO: 9 - DNA, 284 bp, Artificial sequence
  23883765-6
cctatttttataggttaatgtcatgataataatggtttcttagacgtcaggtggcacggtctcatgccgattttcagaccgtagtcaattgacggtggtttgaataaggtatccgtgatgtttcttgcaagtatgttcccacccaaaaggaactttttgatcgtacaattcgttgtaagttcataattgctaaacatttagtctaagggctcggtatatatattagcggaccttgtcattgagttcatgatttttgttcaaggagtatgtgcatgagtattcaa

SEQ ID NO: 10 - DNA, 284 bp, Artificial sequence
  23883741-6
cctatttttataggttaatgtcatgataataatggtttcttagacgtcaggtggcacggtctcatgccccggcgtactgactaaagatattctcattccataaacgttaactgcctaaacatgcatttgcctctgttatagagatggaacgcgctgatgtggacattaaactttgtgttctcgccttcggtggttttaaagggtatgcgtgtgtgattgtgatcaagtacaattaacgaaccatgttcgacaatctttctaggaggaaggggatgagtattcaa

SEQ ID NO: 11 - DNA, 284 bp, Artificial sequence
  23885367-6
cctatttttataggttaatgtcatgataataatggtttcttagacgtcaggtggcacggtctcatgcctgttcattccgagtgtgtgatgattttgtgatggtttgcaaatatgcttcgtgtattttagttgcagatttacttgctgcagacttaagaggccatataattctataaagagcgaggtggcctggtagccggtatctgggtcttggctgtaatttttagaaggtggtgcctagtggcacgacggtacatgggaggaggggggctatgagtattcaa

SEQ ID NO: 12 - DNA, 284 bp, Artificial sequence
  23883717-6
cctatttttataggttaatgtcatgataataatggtttcttagacgtcaggtggcacggtctcatgccagttctctcactttctcagaggtactgttgattctagttacaccagggtgcctattctccttatcgtcctagctctttaattttttgtcagttagtgtattggtcttggttaaggcattatattaattgttatccaggtgccaacaagatgaatactttggtacaaccggaattgttccctcgtgagactataggagcatagggatgagtattcaa

SEQ ID NO: 13 - DNA, 284 bp, Artificial sequence
  23883861-6
cctatttttataggttaatgtcatgataataatggtttcttagacgtcaggtggcacggtctcatgccctttttattttaacagaaagaagacgtatcaatgttgagagccaggtaactgttaatatgggtgtgtctgcttcatcgagccaaggctagcattggtttgtgttttttggtggcaaatgctggaaaggatagtaagatcatcaaaggaggtaacgtgcaaataacgatatgcgttaaggttctattctgggacggagcagagggatgagtattcaa

SEQ ID NO: 14 - DNA, 291 bp, Artificial sequence
  23883693-6
cctatttttataggttaatgtcatgataataatggtttcttagacgtcaggtggcacggtctcatgcctatagcatttttcattggatattatatacttattatgaaacttccccacgttggatgaattcgttgatttcgctacgagactttttgttcgaggtgcttaagataacggtgacaatatggttgtggtgcacaggattagaatttcggactctgatcctcttagggtagtgtgggcaaaatgcttcacagtaagggagcgtggtctctcgtgatgagtattcaa

SEQ ID NO: 15 - DNA, 272 bp, Artificial sequence
  23885085-6
cctatttttataggttaatgtcatgataataatggtttcttagacgtcaggtggcacggtctcatgccgatgttataagatgttgcttatttggtatcatcctatatgtacaccaaccggatacgattttatggaaaactggggttagagtgtgctttaacccattgtgagctggctgcacggaggttgcctattttgatacccgtacagccgtgctcgtgtggactcgtattttaatgatcggagcctaccgctcggccatgagtattcaa

SEQ ID NO: 16 - DNA, 283 bp, Artificial sequence
  23892801-6
cctatttttataggttaatgtcatgataataatggtttcttagacgtcaggtggcacggtctcatgccgcaagagaatttcctgcgaagttcacgttttacattagggtttagtgacttttttggcacagcaccttatttgtgctacgttggtaaaggagctcagtttttcgtagcagagtagttcgaaggtgggcatgttcatacatacttaactccgtgttccggaggtattctacgctgattctccataaatggagtggagcgcgatgatgagtattcaa

SEQ ID NO: 17 - DNA, 284 bp, Artificial sequence
  23883903-6
cctatttttataggttaatgtcatgataataatggtttcttagacgtcaggtggcacggtctcatgcccagcctactattcgctaactaaggcatagtgataccgattagcgaattgttgttgattattccgtttcgtcaaatataattttgctttacaaattggaaatcgtggatcgcttttcattaagggaagaaccaatatgcttcggagggggatgatctgataggtggtgggttaggggactagtgtgagtttgggggaggcaggggatgagtattcaa

SEQ ID NO: 18 - DNA, 308 bp, Artificial sequence
  23887647-6
cctatttttataggttaatgtcatgataataatggtttcttagacgtcaggtggcacggtctgaattcgcggccgcttctagagggtctcatgccgcacggcaactccccgttagaaatgagttagcataccctcatccatccagagacttggtctagtttcttcccaggttgtaatgtaacgtgctattaaaacgaggttgtaaaattatggggaggtaggttatgtcaggagatgtcaatgggttacgcagggaggtcaagccggttatacgcggtgatgccgggagtaatcgtatgagtattcaa

SEQ ID NO: 19 - DNA, 284 bp, Artificial sequence
  23894490-6
cctatttttataggttaatgtcatgataataatggtttcttagacgtcaggtggcacggtctcatgcctctaacggagtgcgatatcgggtcatttcatcctcggccagagtggtgcgtatgggtgtcagtatagggcgtaccgaaatgtatcattttttaagcgagcaaacggtacggattgacaggtcccgtgaaagggagcgacggcgtcgatgtgacgggctcgggaggtctgaatgctggcgggtcgcccggtctaggagagcggtgatgagtattcaa

SEQ ID NO: 20 - DNA, 180 bp, Artificial sequence
  23884065-6
cctatttttataggttaatgtcatgataataatggtttcttagacgtcaggtggcacggtctcatgccattaaaaataattagtgtacggtcgtggcattggctcattttgtatattgaacatgtaccatgaaatcgttcttgcggatgttggaggcggtctcgtcggatgagtattcaa

SEQ ID NO: 21 - DNA, 284 bp, Artificial sequence
  23892918-6
cctatttttataggttaatgtcatgataataatggtttcttagacgtcaggtggcacggtctcatgccaaaactgtgcgggatttgttggagcattcttacagaacttatgacatggatggattgtagattataagaattgggcttctatcttgcctgtaactcgagcggctgtttgcctgtttgggttcatggatgtgagtttgtgtggactttgagacatcggtctgccagggcgttggaagggcgagagacttgtcaaggaggggggtgatgagtattcaa

SEQ ID NO: 22 - DNA, 186 bp, Artificial sequence
  23885328-6
cctatttttataggttaatgtcatgataataatggtttcttagacgtcaggtggcacggtctcatgccctgttgtctttctctatgcacttttttgtatgtatcaaaaaactctaacaaatgtgatcttaccttaattataagctttctgcgcgtcacttatgggagggtggggatgagtattcaa

SEQ ID NO: 23 - DNA, 68 bp, Artificial sequence
  Consensus
cctatttttataggttaatgtcatgataataatggtttcttagacgtcaggtggcacggtctcatgcc

SEQ ID NO: 24 - DNA, 22 bp, Artificial sequence
  BB-Prefix-Fwd
gaattcgcggccgcttctagag

SEQ ID NO: 25 - DNA, 21 bp, Artificial sequence
  BB-Suffix-Rev
ctgcagcggccgctactagta

SEQ ID NO: 26 - DNA, 30 bp, Artificial sequence
  Bsa1-Rev
gggtcccgcggtatcattgcagcactgggg

SEQ ID NO: 27 - DNA, 30 bp, Artificial sequence
  Bsa1-Fwd
aatgataccgcgggacccacgctcaccggc

SEQ ID NO: 28 - DNA, 36 bp, Artificial sequence
  Bsa1-Bla-Rev
  misc_feature (8)..(12): N is any c, g, t or a
cggtctcnnnnnatgagtattcaacatttccgtgtc

SEQ ID NO: 29 - DNA, 33 bp, Artificial sequence
  Bsa1-Bla-Fwd
ggcatgagaccgtgccacctgacgtctaagaaa

SEQ ID NO: 30 - DNA, 22 bp, Artificial sequence
  Bla-Rev
ctttcaccagcgtttctgggtg

SEQ ID NO: 31 - DNA, 18 bp, Artificial sequence
  V-Fwd-pHH100
gggccgagcgcagaagtg

SEQ ID NO: 32 - DNA, 25 bp, Artificial sequence
  V-Rev-pHH100
cgattaagttgggtaacgccagggt

SEQ ID NO: 33 - DNA, 43 bp, Artificial sequence
  I-Fwd-pHH100
ggcgttacccaacttaatcgatggtttctaaaggtgaagaaga

SEQ ID NO: 34 - DNA, 35 bp, Artificial sequence
  I-Rev-pHH100
cttctgcgctcggcccctgagataggtgcctcact

SEQ ID NO: 35 - DNA, 34 bp, Artificial sequence
  Bsa1-mCh-Fwd
  misc_feature (8)..(12): N is c, g, t or a
cggtctcnnnnnatggtttctaaaggtgaagaag

SEQ ID NO: 36 - DNA, 31 bp, Artificial sequence
  Bsa1-mCh-Rev
ggcatgagaccgaagacgaaagggcctcgtg

SEQ ID NO: 37 - DNA, 60 bp, Artificial Sequence
  pKC_fwd_kan
actggtccacctacaacaaagctctcatcaaccgtggctctgatcatctcgcgcagctcg

SEQ ID NO: 38 - DNA, 60 bp, Artificial Sequence
  pKC_rev_kan
gtgcaatgtaacatcagagattttgagacacaacgtggcttgccctcatctgttacgccg

SEQ ID NO: 39 - DNA, 34 bp, Artificial Sequence
  BsaI_Apr5f_new
  misc_feature (13)..(13): n is a, c, g, or t
agttcggtctccnatgtcatcagcggtggagtgc

SEQ ID NO: 40 - DNA, 33 bp, Artificial Sequence
  BsaI_oriV3r
ggcatgagacctcgttccactgagcgtcagacc

SEQ ID NO: 41 - DNA, 20 bp, Artificial Sequence
  5511_F
gcagagcgaggtatgtaggc

SEQ ID NO: 42 - DNA, 20 bp, Artificial Sequence
  234_R
tcgtcgtccagacctgacca

SEQ ID NO: 43 - DNA, 20 bp, Artificial Sequence
  5654_F
aaacaaaccaccgctggtag

SEQ ID NO: 44 - DNA, 20 bp, Artificial Sequence
  apr_5c
acgctacggaaggagctgtg

SEQ ID NO: 45 - DNA, 4 bp, Artificial Sequence
  Pribnow box
ggag

SEQ ID NO: 46 - DNA, 6 bp, Artificial Sequence
  Pribnow sequence
tataat

SEQ ID NO: 47 - DNA, 7 bp, Artificial Sequence
  TATA box consensus
  misc_feature (5)..(5): N is a or t
  misc_feature (7)..(7): N is a or t
tatanan

SEQ ID NO: 48 - DNA, 27 bp, Artificial sequence
  Adapter_02_bla_UpS
  misc_feature (1)..(1): n is a, c, g, or t
natgagtattcaacatttccgtgtcgc

SEQ ID NO: 49 - DNA, 23 bp, Artificial sequence
  Adapter_02_bla_LoS
gcgacacggaaatgttgaatact

SEQ ID NO: 50 - DNA, 26 bp, Artificial sequence
  Adapter_02_kan_UpS
  misc_feature (1)..(1): n is a, c, g, or t
natgattgaacaagatggattgcacg

SEQ ID NO: 51 - DNA, 22 bp, Artificial sequence
  Adapter_02_kan_LoS
cgtgcaatccatcttgttcaat

SEQ ID NO: 52 - DNA, 22 bp, Artificial sequence
  Sc_pENZ004_G_trp_F
atgtctgttattaatttcacag

SEQ ID NO: 53 - DNA, 40 bp, Artificial sequence
  Sc_pENZ004_G_trp_R
ctctagaagcggccgcgaattctacccatctatgctgaag

SEQ ID NO: 54 - DNA, 21 bp, Artificial sequence
  Sc_pENZ004_Hom_F
cgtctatgaggagactgttag

SEQ ID NO: 55 - DNA, 17 bp, Artificial sequence
  Sc_pENZ004_Hom_R
ctcgccaaggcattacc

SEQ ID NO: 56 - DNA, 20 bp, Artificial sequence
  Sc_pENZ004_Seq
cactactctcttcctagtcg

SEQ ID NO: 57 - DNA, 32 bp, Artificial sequence
  Sc_pENZ004_B_trp_F
  misc_feature (9)..(9): n is a, c, g, or t
cggtctcgnatgtctgttattaatttcacagg

SEQ ID NO: 58 - DNA, 30 bp, Artificial sequence
  Sc_pENZ004_B_trp_R
cggtctctggcatacccatctatgctgaag

SEQ ID NO: 59 - DNA, 27 bp, Artificial sequence
  Adapter_Tt_pMK184_kan_UpS
  misc_feature (1)..(1): n is a, c, g, or t
natgaatggaccaataataatgactag

SEQ ID NO: 60 - DNA, 23 bp, Artificial sequence
  Adapter_Tt_pMK184_kan_LoS
ctagtcattattattggtccatt

SEQ ID NO: 61 - DNA, 23 bp, Artificial sequence
  Tt_pMK184_kan_F
atgaatggaccaataataatgac

SEQ ID NO: 62 - DNA, 42 bp, Artificial sequence
  Tt_pMK184_kan_R
ctctagaagcggccgcgaattcgaggctaagccttccctcag

SEQ ID NO: 63 - DNA, 22 bp, Artificial sequence
  Tt_pMK184_seq
atggtagaaatgttgtcggtcc

SEQ ID NO: 64 - DNA, 22 bp, Artificial sequence
  Cg_pXMJ19_Chl_Seq
tgagcaactgactgaaatgcct

SEQ ID NO: 65 - DNA, 40 bp, Artificial sequence
  Cg_pXMJ19_Chl_F
  misc_feature (11)..(11): n is a, c, g, or t
ctcggtctccnatggagaaaaaaatcactggatataccac

SEQ ID NO: 66 - DNA, 35 bp, Artificial sequence
  Cg_pXMJ19_Chl_R
ctggcatgagaccgccccgaagaacgttttccaat

SEQ ID NO: 67 - DNA, 25 bp, Artificial sequence
  Pp_pHH100_mCherry_F
atggtttctaaaggtgaagaagaca

SEQ ID NO: 68 - DNA, 42 bp, Artificial sequence
  Pp_pHH100_mCherry_R
ctctagaagcggccgcgaattcgctgacgccgttggatacac

SEQ ID NO: 69 - DNA, 21 bp, Artificial sequence
  Pp_pHH100_mCherry_Seq
gatgtcagccgggtgtttaac

SEQ ID NO: 70 - DNA, 22 bp, Artificial sequence
  All_biobrick_F
gaattcgcggccgcttctagag

SEQ ID NO: 71 - DNA, 33 bp, Artificial sequence
  library_biobrick_del_F
gaattcgcggccgcttctagagagtctcatgcc

SEQ ID NO: 72 - DNA, 21 bp, Artificial sequence
  library_biobrick_R
ctgcagcggccgctactagta

SEQ ID NO: 73 - DNA, 41 bp, Artificial sequence
  Adapter_pENZ004_trp_UpS
  misc_feature (1)..(1): n is a, c, g, or t
natgtctgttattaatttcacaggtagttctggtccattgg

SEQ ID NO: 74 - DNA, 32 bp, Artificial sequence
  Adapter_pENZ004_trp_LoS
ggaccagaactacctgtgaaattaataacaga

SEQ ID NO: 75 - DNA, 4 bp, Artificial Sequence
  NATG overhang
  misc_feature (1)..(1): n is a, c, g, or t
natg

SEQ ID NO: 76 - DNA, 16 bp, Artificial Sequence
  Consensus 2
ggggatgagtattcaa

SEQ ID NO: 77 - DNA, 211 bp, Corynebacterium glutamicum
tgcccgggctcgagttcaaggattgcgacaatctgttctgtggcagcgtttgagcttatgtgataatctgaattgtaagtgagtatcgtaagggattttcatactagttgagtttacagtaaagttaagtgtgtatcgctagttaactaatcgggacttgaaattctagcaagagtagtgtcccggcacagaacaaggagtgttatgcatg

SEQ ID NO: 78 - DNA, 209 bp, Corynebacterium glutamicum
agtgcccttaataagtccatttgattgttccattatagtctagatacgtcctggtattttgcaattaggattggatgtagtccgtagcagtatttttttatgtgtatagatttacaatgagtgtcgtatattttgcagatgcctccgggattggactggtatcaatattcacggtagctttgagaattatgagtagggaaggacaaatg

SEQ ID NO: 79 - DNA, 213 bp, Corynebacterium glutamicum
agtgccggcctgacaatggatggtattggatcttaattgcagtagctttctgtctgccgattatcaagaatttcttgacaggtgcgaaggtagtcaccctggtgaggcatagtagttcggatgcatattgcaaagagttttcgtggggaagttgcgaatggtcgtaagaatggttgctcaacgtcggtaacttctctacaatgggtatgcatg

SEQ ID NO: 80 - DNA, 213 bp, Corynebacterium glutamicum
agtgccaaagtccatggatttgtagttacgtttattggttgttctcattgggtgactattaagatgcgatccatgatgttttagggctggtatgatatcgagaggttttcatatcatttctatgtatgtccggtgagatctttaatattctgggatcattgatgactccaacacttgatatattgatacgttttaattagtattcaatgaatg

SEQ ID NO: 81 - DNA, 213 bp, Corynebacterium glutamicum
agcatcaacccattgccacatcctactgaaaggatcaccattacgtgttactacatgggtcgctcaccgccgacttccgcatgaccatacgcaaatttcttacctagggccgttcgatcatacttgcaatgcacccaatatgcagtagaatccgatacatcaaaacaccaattatcaatactacataaggacaccaaggattcgaggcaaatg

SEQ ID NO: 82 - DNA, 214 bp, Escherichia coli
tctcctcatttctattgagtcatcctagcattagagttatttgtcagagagtacagtattaagcgattttatggtgtcctaggaggtaggggtatgtacacagttcgtgcttataaaatgtggccttttcctagtgtttacgtgccgttttttctgcgacggtgtccccagatgcattgttttgagcggttgtgctcgagggagttgcataatg

SEQ ID NO: 83 - DNA, 213 bp, Escherichia coli
tgaatgaagcaattgatttaggtcccgcatggataggtaatgctcctcttgccatgtttttgccttcttgaccaagtgggtctatacttttcgttgtaataattgcatctctctggacgaacttgtcgggctattgtaggtttttttccgtgtttgctgtgccttctttttcggaggactaagtgccatacaggtccttggagcagcaacatg

SEQ ID NO: 84 - DNA, 214 bp, Escherichia coli
cttcttagggtgaagtttattatctactcatctgcgttgggggtctcaggtatatgggttttgaggctatatgttgggttataggcatcatttctcctcttcgcgtgttactttgaaaatacgttctacgatatcacctcgaacggcatcggggtcttggctcaccgcaaggatacggcaacctatgttaactagattttggagacggattatg

SEQ ID NO: 85 - DNA, 214 bp, Escherichia coli
ttaattttccaatgggttgcttcatttacattatttatcaacccgtggtaatgtcgttctactttcatctatagtgtgtaatgttattgttttctttctctgttggtggctttttagggggttgtactgtacagcgttctctattatgcgttaaactttgaattcatataggatgaatttatacaaaaagtcacatttaaggagtgttacgatg

SEQ ID NO: 86 - DNA, 214 bp, Escherichia coli
gccaaatgattcctaatatgtctcttatcacgggtcgcgcaacggttactgtcgaggttcgcatatcgttgcacctgtgggttaagagcggtacgtgtgagagtagcacttaatctattccaccttagtacagtgggtatcacttggctttgtcgggttagttcgatcgtgtgagttagatcgctaaagttagtgtggtaggagtatatctatg

SEQ ID NO: 87 - DNA, 214 bp, Pseudomonas putida
aaaaagtaatttgtaaaaaagcagactcacggttgcgattgcacccagggtgttcggggcggtttcttcttggtcatcttatattgaggggggctgtcttgatttatatagttttactgtcctccgatctcctttctcaaatagttcgaagtgtatggtttacttgtttgttatcatgggacgggtctgagtgaggaaatggagaacgacaatg

SEQ ID NO: 88 - DNA, 213 bp, Pseudomonas putida
gggctaagggtaattcttctggatttaaaaaatgcctttttattgtctggttctgtgggaacagccgatgcggtctggttgttctaattgcgcgtcgtttcagcctatgtgctagggtctgtcctagtgcttccaaacaatggtattctctcatttggcactaatggatctgtgattagtacgcggtatgtgggcttagggagcaatataatg

SEQ ID NO: 89 - DNA, 213 bp, Pseudomonas putida
tagcaatattctattttgtgttgcgtatttaatttaatgcagttacgttccccgcttgggatttggccttttgtacattctgtggtttttgctgaggatttgttgcgtgctatgtaacgagggtggtactccaaattgttgtactaaggggtttcttggtatgtaatgggaggttgtatttattctttgtattttgaagggagctctataatg

SEQ ID NO: 90 - DNA, 202 bp, Pseudomonas putida
cgcattttaagcttctataggctggtggagctcctgcggctccatgttgtattattgtggaaaattgtctgaactgacggtaagttttcatgttactgtcgttcggggtatgcccctattatatgatttatgggtagaactagtatttccagcgattttacctcttactgggccacgtatgctattgtggaggctaagtatg

SEQ ID NO: 91 - DNA, 214 bp, Pseudomonas putida
acaggaaatgggagttaggatcacccagcgaattgtgatcattattgcttagatgtatggtatagaatttcttgtttgttttttacctgtgtggttaaaatgccaggaaaggttgaaatgagtcagcactgaccccattgataactgtctgtcgatcggataccgttgactggctgccttctagttcggctgcgttgcgaggagacgacacatg

SEQ ID NO: 92 - DNA, 195 bp, Saccharomyces cerevisiae
aagagtcgcgctcagttttcggttgcttcctggttatctagagggaatttcgatgatgcatttgcctaaatactagctagtgtcaagagatgatattatgtcttatttagacttagcgcggaactaagaattgtcgtttatttactgtctatgaacaatttcgtggactgtgtaatagttgtaactcggtagatg

SEQ ID NO: 93 - DNA, 202 bp, Saccharomyces cerevisiae
gtctgtgtcatgctacaagggtgacggtgcaagtattttgagatcacagtcgtacatgcgtccgcatttcttgaatatttgcactatcacaactatattactgttctgacaaaagtcatcttattttgattaatgttgggggggtgtagtaatggacgcgctagtaatttgtaggtttgtgtcagaaagctcatccgagatg

SEQ ID NO: 94 - DNA, 203 bp, Saccharomyces cerevisiae
tattggtaatagtaccggttgtaaagttgccgctgtcgaacgtggttatttatttggtgttacaatctgggccgcgggttactcttttatctcaagtatttgtgtggttgtcgtttcgcaagctatgtaactcagtctagacgattggactagaggtaggatatttgtcggagattactattgggagtttcttgtagagtatg

SEQ ID NO: 95 - DNA, 203 bp, Saccharomyces cerevisiae
aggcttgccgcagagtgactgatcggtatattcgttattatctaattttcttgtttggcggatggaatacgatagcgttgattgatgttcgtttttcttgtgcagtggcggtttgggtgttaggggtaagtgatcgtgaaagaatcaacggttagatcgaaatgaagtgagattcagtgttggggatggaaatgctaaggatg

SEQ ID NO: 96 - DNA, 203 bp, Saccharomyces cerevisiae
caactggcgcggcaatgttgttacttttaagagttttcttctcgatccttaccaaaggaatggattcttattgagatttaagctggcttatgttgtaagtctgttacaataagtcgtgcgaaggcggtttacgaagaaggtagtctgggagtcctgtacttgtttatcatctattatttgtttttccaagaccggggttgatg

SEQ ID NO: 97 - DNA, 182 bp, Streptomyces albus
tgtgatggaagatattagtgagaagtctctgatgcgaagtttttagttggccccggttgttgctgttatacaatatctaacaatccccgacgagcaatacaccgggttccagcacctgggacatgttttacgcgtctgaaaatatgattagagaggagtagtgagtgtctcgaggactgatg

SEQ ID NO: 98 - DNA, 203 bp, Streptomyces albus
gaacatatgggaggaagatggataccctatgtgttcccggtatgtatcatgtgatatatcggttcaatggcacgagacaacgagagggacaggtctaaaggccattatatgggcggtagcgtatgtttgtgtattcgttaggtcagttgtggtatcaacaattaaagttcaacgtctatgctagtatgggggccaaatagatg

SEQ ID NO: 99 - DNA, 203 bp, Streptomyces albus
ctctgtgttaccagatctttaaggtatagacaggtgtgttcgagcgatggattttttttttcttttaactcattgtagcgctaaaactgttggtcgtatgcgggctattggtacatgtgaaggagtcacaagtctggcaagcaggttgtgtcatgtgtaggtaccagaataagcaggcatagggtagactggaggggattatg

SEQ ID NO: 100 - DNA, 203 bp, Streptomyces albus
tccttacacattggtatttttctcctagttgcctgtctttgcaatcagtactcttgaccgttttttccatggatcaagagttaaagggtgggggtgaatgtactcaaattatggttcagtgggggtaatatccgttctgactgtgaacattagcacagtagtgtggcggacggcggcgttgttggtatagtatgggcttgatg

SEQ ID NO: 101 - DNA, 203 bp, Streptomyces albus
ctaccatggcagggcttttacgtggatatcttctttgagtgtttgcctatcggtttgtgatatgcttagatctatcaggtgtcgacttccgtacttgttggtaatgctcagatcatcaatcgaagggcgaatgttgtacaatcagtatgtgacggtctgagggggacggttttgtatggggcgccggggagttggttagtatg

SEQ ID NO: 102 - DNA, 142 bp, Streptomyces lividans
ttctcttatcaataacttagtaaagaccttgggagacaatcgggagatgccgtgacgtgggttgcgagttgggtacgtcctttacggaaggtcatgcgtggtaagttgcggtgcgtatggagataagataaggtggagcatg

SEQ ID NO: 103 - DNA, 203 bp, Streptomyces lividans; identical to SEQ ID NO: 101

SEQ ID NO: 104 - DNA, 203 bp, Streptomyces lividans
tcggtcgcggagtgaatatcccctgttgcgaggtataagtgtattcttgcgtattgggagattgtggtcgcagatcgtatgcttgatctggtgtcaatttagggaagtaattacctgacgagaggtcgttgtctcggtttgagtataaggctatatgtgtgttttgcgtgttggggccaagatgcgcataagatgatggtatg

SEQ ID NO: 105 - DNA, 203 bp, Streptomyces lividans
gaaatatctctctgtcttgaacggggttgtaagggatataattcctaggatcgttgactctcccggacggttctttcgggcggttttctcgttgttccaggttgggagtgttctggcttttgaacctcttgcatgctgtcaggggtggttgtgtataatcgctgagttgttcatacaaattggttgggagtgttcgcaagatg

SEQ ID NO: 106 - DNA, 142 bp, Streptomyces lividans; identical to SEQ ID NO: 102

SEQ ID NO: 107 - DNA, 214 bp, Thermus thermophilus
acagcgtttgcaatggtaactcgcgtttatgctatacttgaaagcgaactagcattagaactctaaaaggtgattgtaagtgacgatggagtgttaatcttaggggggggacgcgattagattaggcgcatccgtctagttaagtgttgggccgtcagaggacggcaagaacttcggtggttagattggtcatgtgagcaggagtcatgcaatg

SEQ ID NO: 108 - DNA, 214 bp, Thermus thermophilus
acttctggcgtactttcatgcagtcagaatacgatccttggaaaactacatgcttaataaagggagaaaagggtcatgttcttcggaaaggtcgaacattgttgatgtcgattggtgacttgttgttttgatttatgataggttgtatgggtcgtgggctgagaaggctctagacttgtcgagtagtacggtcatgagggggagcgtgtatatg

SEQ ID NO: 109 - DNA, 214 bp, Thermus thermophilus
gttggtcaggtatactttgcttaacgtttggtttattttctttattttttaagatgtcctaggttaaattattctgctttttcttgcggattagggcgttgctcgtttaggatagtaattttataatgaggttgtgcgtagcggccatgatgtgttccatgtatgacactagaatggggtgagcgggcctgttatggtgcggagtgatcttatg

SEQ ID NO: 110 - DNA, 214 bp, Thermus thermophilus
ttaccatttcagccgcggtcttttataattcggtagcggtgcgttacgggggttaaggtgacggtcgtcacgtggttggagacacggttcgggattgtgttctatcattgatgggggtagaagggagtacatttagtggtcgtcctttggctgcctggtttgagggaaaataattgctgactgctgtagcgggaaaggccggaggggctggatg

SEQ ID NO: 111 - DNA, 214 bp, Thermus thermophilus
aggtggggtacgctgtttttatactttgttggtctttattgtgttatagtgatcggaagataggaaaaatagttgtgtgatatgctgggatttgtgtggtggttgtctagcaggctttgcagttagacgtggacatttggtgggggggcgtaccgggtcagggttgtgtggtaagtaattgcgggcttaaattcggttgtggagagggtgaatg

(19) United States
(12) Patent Application Publication
(10) Pub. No.: US 2020/0102557 A1
(43) Pub. Date: Apr. 2, 2020

(54) MODULATION OF GENE EXPRESSION

(71) Applicant: Norwegian University of Science and Technology (NTNU), Trondheim (NO)

(72) Inventors: Martin HOHMANN-MARRIOTT, Dunedin (NZ); Rahmi LALE, Heimda (NO)

(21) Appl. No.: 16/498,053

(22) PCT Filed: Mar. 29, 2018
(86) PCT No.: PCT/EP2018/058227

§ 371 Date: Sep. 26, 2019

(30) Foreign Application Priority Data
Mar. 30, 2017 (GB) 1705121.0

(51) Int. Cl. C12N 15/10; C12N 15/70

(52) U.S. Cl. CPC C12N 15/1086; C12N 15/70

(57) ABSTRACT

The present invention provides methods for identifying a factor which modulates gene expression comprising providing a library of at least 1×10⁶ nucleic acid molecules each comprising a stochastic sequence of at least 50 nucleotides, introducing said nucleic acid molecules into nucleic acid constructs which comprise a reporter sequence and which do not comprise a further separate sequence upstream of the reporter sequence that can modulate expression of the reporter sequence to generate test constructs, introducing test constructs into host cells and assessing expression of the reporter sequence, thereby to identify a factor which modulates gene expression. Libraries of nucleic acid molecules, test constructs and host cells for use in such methods are also provided.

The present invention relates to the identification of factors which influence, or modulate, gene expression. Such factors may include genetic elements that modulate gene expression at the level of transcription and/or translation, as well as conditions or stimuli to which such genetic elements may respond. Specifically, the present invention provides methods and compositions for discovering or identifying elements upstream of a gene which can affect expression of the gene and/or which are responsive to conditions which may modulate expression of the gene, including both internal and external stimuli which may affect expression of the gene in a host cell.

Gene expression is controlled to a large extent by nucleotide sequences which are situated upstream of the coding region of that gene. These sequences, which are also known as elements which can modulate gene expression (which terms are used interchangeably herein) can affect expression of the gene/production of the protein in various different ways, including both at the level of transcription of the coding sequence into mRNA and at the level of translation of the mRNA into protein, to result in production of the encoded protein. Gene expression may thus alternatively be termed protein expression and includes all aspects of expression, or production, of the protein from the gene to the primary encoded protein product. Gene expression-modulating elements may serve as binding sites for cellular components which can mediate, induce or repress gene transcription or which can drive or modulate (i.e. affect in any way) protein synthesis following formation of a transcript of the gene, as well as sequences which may affect the transcription and/or translation process in any way. Thus, the interaction between these cellular components and the sequences upstream of a gene largely determines the extent to which a gene is expressed, although the sequences may themselves also have an effect, e.g. on transcript stability. Such sequences include notably enhancers, promoters or promoter elements (i.e. parts or constituent sequences of promoters) 5′ untranslated regions and other regulatory sequences (enhancers etc.), or binding sites for regulatory molecules, ribosomes, DNA polymerases or RNA polymerases, or other factors which may affect, or modulate expression of the protein.

A large number of sequences which influence gene expression in a wide range of different organisms are known in the art and are used to regulate gene expression in, for example, methods of protein production and gene expression studies. However, regulatory sequences are typically both smaller in size, and less well conserved between different species than the coding regions which they regulate, and thus the identification of regulatory sequences from genome databases alone cannot be performed as readily as for coding regions or structural genes.

Expanding the number of available regulatory sequences is a long-term goal in the fields of gene expression, recombinant protein production and synthetic biology, and a large number of studies which aim to produce and characterise new regulatory sequences using a variety of different techniques have been performed. For example, a library of random nucleic acid sequences was tested in conjunction with the CMV promoter in a HeLa expression system, in order to identify sequences with enhancer activity (Schlabach et al. 2010. PNAS 107, 2538-2543), and consensus sequences to known cAMP response element (CRE) and P53 binding site sequences were identified in such a method. However, this approach relies on the activity of an existing promoter to drive gene expression when testing the effects of the random sequences, and thus this approach will tend to identify only those enhancers which work in combination with a particular promoter sequence. Alternative approaches, which seek to produce and identify new promoter sequences or modify the function of an existing promoter sequence, are based on altering promoter properties by ‘evolving’ known sequences (U.S. Pat. No. 6,337,186, WO 98/01581), or fragmenting and reassembling known promoters (WO 02/068692, US 2005/0227246, US 2005/0003354). However, these methods use known regulatory sequences as a starting point for identifying new sequences, and thus are limited in the extent to which they are capable of identifying new elements which modulate gene expression.

It is clear, therefore, that any elements identified in these methods would have been identified in the particular context of these known sequences, rather than from a truly stochastic starting point in the absence of any other elements which modulate gene expression.

By contrast, the present invention involves a method of identifying an element which modulates gene expression, which is not constrained by any of the limitations described above. Rather, the present invention provides a method of identifying an element which modulates gene expression which is not limited to identifying variants of known elements, or identifying elements which modulate gene expression only in combination with a known element. The present invention provides a method which screens stochastic nucleic acid molecules for their ability to modulate expression of a gene in a way which can be independent of any other genetic elements, using a cell-based expression system, and allows nucleic acid molecules which might comprise a sequence for an element which modulates gene expression to be identified.

More generally, the method may be used to identify any factor which modulates gene expression, including for example the identification of a particular condition which modulates gene expression, e.g. the presence of an internal or external stimulus, or factor, in the host cell, which has an effect on gene expression.

Thus a library of stochastic nucleic acid molecules may be provided and used to prepare a library of clones harbouring stochastic molecules, which library of clones may then be screened for their ability to modulate gene expression in the presence of a test condition, and in this way different test conditions may be screened for their possible effect on gene expression. If such a test condition is identified, then the nucleic acid molecule which mediates a response to it (i.e. which responds to the test condition with an effect on gene expression levels) may be identified,

As described in greater detail below, a further advantage provided by the method of the present invention is that the stochastic sequence provided in each nucleic acid molecule is longer than those used in the methods of the prior art. Whereas prior art methods which screened stochastic sequences screened shorter sequences, in order to identify individual elements (e.g. repeats of 10 nucleotide random sequences in combination with a known promoter, as described in Schlabach et al. ibid), the present invention utilises longer stochastic sequences. This allows longer elements, combinations of elements, and/or elements found at a greater distance upstream of the coding region of a gene to be identified, which would not be possible using the methods described in the prior art.

Thus, the present invention allows potential new elements to be screened for their ability to modulate gene expression without the screen being limited to sequences which are based on, or function in combination with, known elements. Put another way, the present invention allows a deeper screen of elements which modulate gene expression to be performed, and thereby provides greater opportunities for new such elements to be identified than the methods of the prior art. Thus, the present invention allows elements which would not be discovered using the methods of the prior art to be identified. Furthermore, due to the extended length of the stochastic sequence used in the methods of the present invention, elements may be discovered in a combinatorial manner, i.e. new combinations of elements or combinations of new elements may be identified.

The present invention is based on the concept of providing and using a library of nucleic acid molecules which is large enough to encompass all, or almost all or at least a significant proportion of, possible sequence variations for a nucleic acid molecule of given length. In this way, previously untested nucleic acid sequences may be screened in an assay for measuring gene expression, thereby allowing new factors which modulate gene expression to be identified. By introducing the library of nucleic acid molecules into a host cell (more particularly, in the context of a test construct in which the nucleic acid molecules are operably linked to a reporter sequence), a library of host cells may be provided, which comprise a stochastic selection (or subset) of the initial nucleic acid library. In this way, a wider range of nucleic acid sequences may be screened, and thus the probability of identifying a factor which modulates gene expression (e.g. by recruiting a wider range of native elements and native factors affecting expression (e.g. transcription factors)) is increased. Thus, the present invention attempts to represent in the initial library of nucleic acid molecules as complete as possible, or as practicable, a range of stochastic sequences, and whilst for various reasons (e.g. to do with synthesis and recovery of synthesised molecules) not every theoretically possible nucleic acid sequence may be represented in the initial nucleic acid molecule library (i.e. the nucleic acid molecule library is not necessarily absolute in terms of stochastic variation), the library covers a significant, or very large portion of stochastic sequence variations, such that the solution space of the library is large enough in theory to allow the identification of many, or most, if not all, genetic elements which may occur in nature, or at least in a given host cell.

Accordingly, the present invention provides a method of identifying a factor which modulates gene expression, said method comprising:
(a) providing a library of at least 1×10⁶ nucleic acid molecules, wherein each nucleic acid molecule comprises a stochastic sequence of at least 50 nucleotides, optionally wherein each nucleic acid molecule also comprises an identical pre-determined and fixed sequence either adjacent or within the stochastic sequence. wherein said pre-determined and fixed sequence does not include a promoter sequence itself capable of initiating transcription of a gene;
(b) introducing the nucleic acid molecules from said nucleic acid library of (a) into nucleic acid constructs to prepare a library of test constructs, wherein each test construct comprises a nucleic acid molecule of part (a) upstream of a reporter sequence, and wherein each test construct does not comprise a further separate sequence (that is, separate to the nucleic acid molecule which is introduced) upstream of the reporter sequence that can modulate expression of the reporter sequence;
(c) introducing test constructs from said test construct library of (b) into host cells to prepare a library of host cells, wherein host cells from the host cell library comprise a test construct;
(d) assessing expression of the reporter sequence in host cells from the library of host cells, optionally under a test condition; and
(e) based on the level of expression of the reporter sequence, identifying a factor which modulates gene expression.

The factor can be any factor which modulates gene expression, and may for example be a factor contained in the nucleic acid molecule (i.e. a genetic element, or regulatory sequence), or in the host cell (e.g. a transcription factor or other regulatory or signalling molecule), i.e. an internal factor, or an external factor, e.g. a particular test condition to which the host cells are exposed or subjected for the assessment of expression. This may be for example the presence of a substance or molecule (e.g. exogenously added to the cell), or any other condition to which the host cell is subjected e.g. any environmental condition, such as light, temperature, pH etc.

The factor may thus be a genetic element, a regulatory molecule present in the host cell or a condition (more particularly an external condition) to which the host cell is subjected for the assessment of gene expression. As will be described in more detail below, the host cell may be subjected to the condition before and/or during the step of assessing expression of the reporter sequence. This step may take place by incubating the host cells and determining or assessing expression of the reporter sequence. Particularly the host cells may be incubated under conditions in which gene expression may take place, i.e. conditions conducive or permissive to gene expression. These may be conditions which permit or allow gene expression in general, e.g. under which growth and/or viability of the host cell is maintained, for example conditions which permit growth. Thus the conditions may be standard growth conditions for the host cell, or for cells in general. Alternatively, the conditions may be a test condition, e.g. a particular or specific condition which is being assessed for its effect on gene expression, or a particular or specific condition which affects, or may affect, gene expression, e.g. the presence of an inducer or repressor molecule etc.

Step (e) may thus comprise identifying the nucleic acid molecule of the expression vector from a host cell having a level of expression of the reporter sequence which is of interest and/or identifying a test condition under which a host cell expresses the reporter sequence at a level of interest, thereby to identify a factor which modulates gene expression.

The nucleic acid molecule may contain a genetic element which modulates gene expression, e.g. a sequence which modulates gene expression or which interacts with a molecule which modulates gene expression, or a binding site for a molecule which modulates gene expression, e.g. a regulatory molecule or factor present in the host cell or in the test condition.

In one embodiment the factor is a genetic element. According to this embodiment the invention more particularly provides a method of identifying an element which modulates gene expression, said method comprising:
(a) providing a library of at least 1×10⁶ nucleic acid molecules, wherein each nucleic acid molecule comprises a stochastic sequence of at least 50 nucleotides, optionally wherein each nucleic acid molecule also comprises an identical pre-determined and fixed sequence either adjacent or within the stochastic sequence, wherein said pre-determined and fixed sequence does not include a promoter sequence itself capable of initiating transcription of a gene;
(b) introducing nucleic acid molecules from said nucleic acid library of (a) into nucleic acid constructs to prepare a library of test constructs, wherein each test construct comprises a nucleic acid molecule of part (a) upstream of a reporter sequence, and wherein each test construct does not comprise a further separate sequence upstream of the reporter sequence that can modulate expression of the reporter sequence;
(c) introducing test constructs from said test construct library of (b) into host cells to prepare a library of host cells, wherein host cells from the host cell library comprise a test construct;
(d) assessing expression of the reporter sequence in host cells from the library of host cells; and
(e) identifying the nucleic acid molecule of the test construct from a host cell having a level of expression of the reporter sequence that is of interest, thereby to identify an element which modulates gene expression.

In another aspect, the invention provides a method of identifying a condition which modulates gene expression, said method comprising:
(a) providing a library of at least 1×10⁶ nucleic acid molecules, wherein each nucleic acid molecule comprises a stochastic sequence of at least 50 nucleotides, optionally wherein each nucleic acid molecule also comprises an identical pre-determined and fixed sequence either adjacent or within the stochastic sequence, wherein said pre-determined and fixed sequence does not include a promoter sequence itself capable of initiating transcription of a gene;
(b) introducing nucleic acid molecules from said nucleic acid library of (a) into nucleic acid constructs to prepare a library of test constructs, wherein each test construct comprises a nucleic acid molecule of part (a) upstream of a reporter sequence, and wherein each test construct does not comprise a further separate sequence upstream of the reporter sequence that can modulate expression of the reporter sequence;
(c) introducing test constructs from said test construct library of (b) into host cells to prepare a library of vectors into host cells, wherein host cells from the host cell library comprise a test construct;
(d) assessing expression of the reporter sequence in host cells from the library of host cells under a test condition; and
(e) identifying whether the test condition modulates expression of the reporter sequence of a host cell, thereby identifying said test condition as a condition which modulates gene expression.

In a further aspect, the invention also provides a library of nucleic acid molecules for use in a method as defined above for identifying a factor which modulates gene expression, wherein said library comprises at least 1×10⁶ nucleic acid molecules, wherein each nucleic acid molecule comprises a stochastic sequence of at least 50 nucleotides, optionally wherein each nucleic acid molecule also comprises an identical pre-determined and fixed sequence either adjacent or within the stochastic sequence, wherein said pre-determined and fixed sequence does not include a promoter sequence itself capable of initiating transcription of a gene.

In a particular embodiment the nucleic acid molecules are double-stranded.

Another aspect of the invention provides a library of test constructs, wherein said library comprises at least 1×10⁶ test constructs, each of which comprises a different nucleic acid molecule positioned upstream of a reporter sequence, wherein each nucleic acid molecule comprises a stochastic sequence of at least 50 nucleotides, optionally wherein each nucleic acid molecule also comprises an identical pre-determined and fixed sequence either adjacent or within the stochastic sequence, wherein said pre-determined and fixed sequence does not include a promoter sequence itself capable of initiating transcription of a gene, and wherein each test construct does not comprise a further separate sequence upstream of the reporter sequence that can modulate expression of the reporter sequence.

The reporter sequence is advantageously the same in each test construct in the library.

In yet another aspect, the present invention provides a library of host cells produced by introducing test constructs from the test construct library of the present invention into host cells, wherein each host cell from the host cell library comprises a test construct.

Each cell in the library of host cells therefore comprises a test construct, and in an ideal situation, each cell in the library of host cells would comprise a different test construct which itself would ideally comprise a different nucleic acid molecule). However, although rare, it is not precluded that two or more cells in the cell library may receive the same test construct. Nonetheless, the preparation of the cell library is carried out such that different host cells (or more particularly different host cell clones) are expected, or intended, to carry a different test construct.

As will be described in more detail below, for various reasons not all of the molecules in the nucleic acid molecule library will be represented in the cell library, and the size of the host cell library will generally be less than the size of the nucleic acid molecule library.

Advantageously, the library of host cells comprises at least 10⁴ host cells, or at least 10⁵, 10⁶, 10⁷ 10⁸, 10⁹, 10¹⁰, 10¹¹ or 10¹² host cells.

The term “factor” is broadly defined herein to include any factor which may modulate gene expression, as discussed above, and thus includes both physical entities, e.g. a nucleic acid sequence, or any molecule, as well as a condition to which a cell expressing a gene may be exposed, e.g. an environmental or cell culture condition. As discussed above, a factor may be an element which is present in the nucleic acid molecule, which may itself modulate gene expression, or which may interact with another molecule or sequence to modulate gene expression, including a molecule present in or produced by the host cell, or to which the host cell is exposed, or it may be a molecule present in the host cell e.g. a regulatory or signalling molecule, or it may be an external or exogenously added molecule. A condition which may modulate gene expression may include the presence of a molecule or substance which may modulate gene expression, or it may be a condition such as temperature, light or other radiation, pH, osmotic or ionic strength etc. An element may thus be directly or indirectly responsive to a condition which modulates gene expression. A factor may thus be an external or internal stimulus which modulates gene expression or an element which responds to an external or internal stimulus which modulates gene expression. An internal stimulus may be regarded as a stimulus which is present in a host cell (e.g. a regulatory or signalling molecule) and an external stimulus may be regarded as an exogenous stimulus which is added to the host cell (i.e. with which the host cell may be contacted), or to which the host cell may be subject or exposed. A condition may accordingly be regarded as being or as comprising an external stimulus which modulates gene expression. An element may include an element which is responsive, directly or indirectly, to a stimulus.

An “element” may particularly be regarded as a sequence in the nucleic acid molecule which modulates gene expression. It may thus be more particularly defined as a genetic element. For example it may be a regulatory sequence or a sequence which interacts with or binds to a regulatory molecule which modulates gene expression. Elements are discussed in more detail below.

The term “modulates” includes any effect on the presence, absence or level or amount or rate of gene expression, including at the level of transcription of the gene and/or translation of the transcript into protein. The term “modulates” may accordingly alternatively be defined as “affects” or “influences” and includes an effect in mediating gene expression, as well as down- or up-regulating the level or amount or rate of gene expression, e.g. mediating transcription and/or translation, as well as increasing and/or reducing the level, amount or rate of transcription and/or translation.

The term “stochastic sequence” means a sequence with a stochastic distribution of nucleotides. That is, the sequence is not predictable or pre-determined, or in other words is based on a stochastic probability of nucleotide distribution in the sequence. A stochastic sequence may be randomly determined, but it is not required for the sequence to be truly random. A stochastic sequence may thus have or comprise a bias for different nucleotides, i.e. it may comprise different nucleotide ratios (i.e. different from A:T:G:C=1:1:1:1).

Surprisingly, the approach of the present invention, which neither modifies known elements which modulate gene expression, nor requires stochastic nucleic acid molecules to be tested in combination with any known elements, has been found to allow the identification of elements which modulate gene expression from within a library of nucleic acid molecules with stochastic sequences. It will be seen, therefore, that the method of the present invention allows the identification of an element which modulates gene expression without requiring any prior knowledge of a regulatory sequence, or the use of any such known sequence in conjunction with a stochastic nucleic acid sequence. This therefore allows a far greater number of different sequences than previously possible in the methods of the prior art to be generated and screened for possible modulatory activity, and thus increases the likelihood that a further element which modulates gene expression can be found.

The test constructs do not comprise further separate sequences upstream of the reporter sequence that can modulate expression of the reporter sequence which are not provided as part of or within the stochastic sequence and/or any pre-determined and fixed sequence situated within or adjacent thereto. In other words, the test constructs do not contain further sequences separate from the stochastic sequence, or further sequences separate from any pre-determined and fixed sequence situated either adjacent or within the stochastic sequence. In this sense, the nucleic acid constructs which are used in the generation of a library of test constructs do not comprise further separate sequences upstream of the reporter sequence found therein that can modulate expression of the reporter sequence, i.e. they do not themselves comprise sequences upstream of the reporter sequence that can modulate expression of the reporter sequence, and no such further separate sequences are additionally provided in or introduced into test constructs in the present invention. Put another way, a test construct may only comprise sequences that can modulate expression of the reporter sequence upstream of the reporter sequence which are provided in a stochastic sequence or in a pre-determined and fixed sequence situated adjacent or within the stochastic sequence, and does not comprise any further such sequences. Thus, such sequences are not provided in a nucleic acid construct used in the generation of a test construct, or separately introduced into a nucleic acid construct before or simultaneously with a nucleic acid molecule, or in a test construct after a nucleic acid molecule has been inserted into a nucleic acid construct.

The nucleic acid molecules provided in step (a) of the methods of the present invention comprise or have stochastic sequences which are screened or tested for any ability to modulate gene expression. In other words, the stochastic sequences may be thought of as test sequences, or as part of test sequences. More particularly, the nucleic acid molecule comprises a test sequence which is assessed for its ability to modulate gene expression (or for its effect on gene expression). The test sequence comprises the stochastic sequence and optionally a fixed pre-determined sequence. Accordingly, in the methods of the present invention, nucleic acid molecules comprising stochastic sequences (i.e. test sequences) are screened for gene modulatory ability, thereby to identify a factor, e.g. an element, which modulates gene expression.

The provision of a library of nucleic acid molecules comprising stochastic sequences may provide a representative sample of all, or almost all available sequences, or at least a substantial proportion thereof, and thus, the use of such a nucleic acid library in the methods of the present invention allows a representative sample of the available nucleic acid molecules to be screened in order to identify a factor which modulates gene expression. It will be seen, however, that in the methods of the present invention, it is not necessary to screen the entire nucleic acid library provided in step (a) of the methods of the present invention in the cell-based screening method of the invention in order to identify a factor which modulates gene expression. Thus, only a fraction (or a portion, subset or a selection) of the nucleic acid molecules may be screened according to the methods of the present invention. The library of test constructs may therefore comprise fewer members than the library of nucleic acid molecules, and the library of host cells may comprise fewer members than the library of test constructs (and thus than the library of nucleic acid molecules).

Thus, not every member of the nucleic acid library may be introduced into a test construct, and not every member of the test construct library may be introduced into a host cell. In other words, not every member of the nucleic acid library may have its own corresponding test construct or host cell. Put another way, the test construct library may represent a selection (or a sample) of the nucleic acid molecules, and thus a selection (or subset) of the stochastic sequences, provided in step (a) of the methods of the present invention. In other words, the test construct library may represent a stochastic selection of the nucleic acid library, or alternatively, may represent a random selection of the nucleic acid library. Thus, in certain embodiments no directed, or no predetermined, selection takes place in the generation of the test construct library and/or host cell library. In certain embodiments, such a reduction in the number of members of the respective libraries during the successive steps of the present invention may be a deliberate choice, i.e. it may only be desirable or practical for only a portion of the nucleic acid molecules of the nucleic acid library of step (a) to be introduced into nucleic acid constructs in step (b), and/or only a portion of the test constructs of the test construct library of step (b) to be introduced into host cells in step (c).

However, it will also be apparent to the skilled person that mechanisms for introducing nucleic acid molecules from a nucleic acid library into nucleic acid constructs, and mechanisms for introducing test constructs into host cells may not be 100% efficient. Thus, even where a nucleic acid construct is contacted with the nucleic acid library (i.e. the entire nucleic acid library) of step (a), and/or where host cells are contacted with the test construct library of step (b), this may not result in every nucleic acid molecule from the nucleic acid library being introduced into its own respective test construct, and its own respective host cell.

The test construct library may therefore comprise less than 100% of the nucleic acid library. In certain embodiments, therefore, the test construct library may comprise less than 99%, 98%, 97%, 96%, 95%, 90%, 85%, 80%, 75%, 70%, 65%, 60%, 55%, 50%, 45%, 40%, 35%, or 30% of the nucleic acid library. Expressed another way, the test construct library may comprise at least 0.1%, 0.2%, 0.5%, 1%, 2%, 5%, 10% or 20% of the nucleic acid library. Alternatively, the test construct library may comprise at least 1 in 2, 1 in 4, 1 in 6, 1 in 8 or 1 in 10 of the nucleic acid library, or may comprise at least 1 in 100, 1 in 1,000, 1 in 10,000, 1 in 100,000 or 1 in 1,000,000 of the nucleic acid library, or at least 1 in 10⁷, 1 in 10⁸, 1 in 10⁹, 1 in 10¹⁰, 1 in 10¹¹ or 1 in 10¹² of the nucleic acid library, or less

Similarly, the host cell library may comprise less than 100% of the test construct library. The host cell may therefore comprise less than 99%, 98%, 97%, 96%, 95%, 90%, 85%, 80%, 75%, 70%, 65%, 60%, 55%, 50%, 45%, 40%, 35%, or 30% of the test construct library. Put another way, the host cell library may comprise at least 0.1%, 0.2%, 0.5%, 1%, 2%, 5%, 10% or 20% of the test construct library Alternatively, the host cell library may comprise at least 1 in 2, 1 in 4, 1 in 6, 1 in 8 or 1 in 10 of the test construct library, or may comprise at least 1 in 100, 1 in 1,000, 1 in 10,000, 1 in 100,000 or 1 in 1,000,000 of the test construct library, or at least 1 in 10⁷, 1 in 10⁸, 1 in 10⁹, 1 in 10¹⁰, 1 in 10¹¹ or 1 in 10¹² of the test construct library, or less.

Generally speaking, with nucleic acid molecule/test construct libraries of very large size, it is the case that the cell library will for reasons of practical reality, or practical necessity, contain a number of members which may be several orders of magnitude less than that of the nucleic acid molecule/test construct library from which it is prepared. This is due to constraints imposed on and by available methods for generating cell libraries, for example for introducing nucleic acid molecules into host cells and recovering host cells which comprise the introduced molecules/constructs. What is important according to the present invention is that a large initial library of nucleic acid molecules is prepared, and that initial library is used to prepare the test constructs and the host cell library. In this way, as discussed above, the cell library can be seen to be representative of a stochastic nucleic acid molecule library, i.e. representative of a stochastic selection of nucleic acid sequences.

Furthermore, and finally, it is not necessary to assess the level of gene expression in every host cell in the host cell library, and it is only required that the level of gene expression is assessed in at least some of the host cells from the host cell library. Thus, where it may not be possible or practical to assess the level of gene expression in every cell in the host cell library, only a subset or selection of the host cells from the host cell library need be assessed in this way.

Nevertheless, the strength of the method of the present invention is the ability to generate a large number of nucleic acid molecules comprising stochastic sequences, and screening said library (or more particularly, a selection of said library, as described above) in order to identify a factor which modulates gene expression. As will be seen from the representative sequences identified in a range of different microorganisms in the Examples provided below, sequences may be identified which can modulate gene expression which do not align well with one-another, either across their entire length or within particular regions. This is indicative of a large sequence space being searchable in the methods of the present invention, and provides benefits in allowing a wider range of test sequences to be tested in order to identify a factor which modulates gene expression.

It is widely-known in the art that for a nucleic acid molecule of “n” nucleotides in length, the number of possible sequences available is 4ⁿ, and thus rises exponentially as the length of the nucleic acid molecule is increased. A nucleic acid molecule of 10 nucleotides in length therefore has approximately 1×10⁶ possible permutations, and the addition of a further 5 nucleotides increases this number by a factor of 10³.

The present invention therefore requires a large number of nucleic acid molecules to be provided. However, it is not necessary for every possible sequence to be provided in order for the methods of the present invention to be performed. Similarly, it is not necessary for every sequence that is provided to be screened. Instead, it is merely required that a large, yet representative sample of all possible nucleic acid sequences is screened (i.e. introduced into host cells via test constructs, and gene expression assessed), in order to allow the identification of elements or other factors, as outlined above. In preferred embodiments, therefore at least 1×10⁴, 10⁵, 10⁶, 10⁷ 108, 10⁹, 10¹⁰, 10¹¹ or 10¹² nucleic acid sequences are screened via introduction into host cells. In other words, in preferred embodiments, the library of host cells comprises at least 10⁴, 10⁵, 10⁶, 10⁷ 10⁸, 10⁹, 10¹⁰, 10¹¹ or 10¹² host cells, or at least this many host cells from a host cell library are screened in the methods of the present invention.

Thus, as a minimum, the library of nucleic acid molecules comprises at least 1×10⁶ nucleic acid molecules, but more preferably comprises at least 1×10⁹, 1×10¹², 1×10¹⁵, or 1×10¹⁸ nucleic acid molecules, up to a maximum of 4ⁿ, where n is the length of the longest nucleic acid molecules in the sample. The number of nucleic acid molecules which form the library may be selected based on the length of the nucleic acid molecules, and it would be evident that a greater number of nucleic acid molecules should preferably be used as the length of the nucleic acid molecules is increased. In one embodiment the nucleic acid library comprises at least 4⁵⁰ molecules. However, whilst the nucleic acid library may comprise up to 4ⁿ nucleic acid molecules, it may not be possible to provide this number of nucleic acid molecules as certain physical and chemical factors may be limiting on the number of nucleic acid molecules which may be provided. For example, the solubility of nucleic acid molecules in water or the number of initiation sites available in the synthesis of the nucleic acid molecules may be limiting on the number of nucleic acid molecules which may be provided in the nucleic acid library.

The present invention requires the nucleic acid molecules which form the library which is investigated in order to identify an element which modulates gene expression to be of sufficient length to accommodate at least a promoter and at least one additional element which can modulate gene expression. Put another way, the nucleic acid molecules, or more particularly the stochastic or test sequences thereof, are of sufficient size to allow such sequences to be formed within and from the stochastic sequence and the optional fixed sequence. The shortest known promoter—the T7 RNA polymerase promoter—is 30 nucleotides in length, and 5′ UTRs, for example are generally at least about 20 nucleotides in length, and typically are in the range of 20-30 nucleotides in length. Thus, each nucleic acid molecule, or more particularly each test sequence or each stochastic sequence is at least 50 nucleotides in length, and may comprise at least 60, 70, 80, 90, 100, 110, 120, 130, 140, 150, 160, 170, 180, 190 or 200 nucleotides. It is not, however, essential that the nucleic acid molecules in the library are of uniform length and/or comprise a stochastic sequence of the same length, and thus a mixture of different length nucleic acid molecules may be provided, provided each is above the minimum length described above. Thus the stochastic or test sequences may be of different lengths, provided the stochastic sequence is at least 50 nucleotides in length. In one embodiment the stochastic sequences are each the same length, and preferably in such an embodiment the test sequences and nucleic acid molecules are all the same length.

Optionally, some (i.e. a subset, or a selection) or all of the nucleic acid molecules which form the library in the above method may comprise an identical pre-determined and fixed sequence. As noted above, where present this is situated within the test sequence of the nucleic acid molecules. Thus, in certain embodiments, the fixed sequence may be situated within the stochastic sequence of the nucleic acid molecules, and thus the nucleic acid molecules may comprise a fixed sequence flanked on both sides by respective portions of the stochastic sequence. Alternatively, the fixed sequence may be situated at the 5′ or 3′ end of the stochastic sequence, and thus be adjacent to the stochastic sequence.

The fixed sequence may have one or more functions which enable or facilitate gene expression, e.g. which are necessary to allow the efficient transcription and/or translation of a gene. Thus, any nucleic acid molecule which comprises the fixed sequence may comprise a sequence necessary to allow transcription and/or translation of a gene to take place. The provision of a fixed sequence may therefore reduce the total number of nucleic acid molecules which need to be provided in order to identify an element which modulates gene expression, as each nucleic acid molecule comprising such a sequence will already comprise a necessary component that allows expression of a gene to take place. Thus, the provision of a fixed sequence removes the requirement for such a sequence to arise in the stochastic sequence of a nucleic acid molecule, and thus allows a greater number of stochastic sequences to be tested for whether they comprise an element which modulates gene expression.

However, a fixed sequence should not itself be sufficient for initiating transcription and/or translation. Thus, whilst the fixed sequence may be a sequence necessary for transcription and/or translation of a gene, it is not sufficient therefor, and thus further sequences (i.e. elements) will be required in order for expression of a gene to take place. In particular, a fixed sequence is not a sequence which by itself is capable of initiating transcription, i.e. it cannot function as a promoter by itself. In other words the fixed sequence is not a promoter, or more particularly it is not the minimal sequence of a promoter (i.e. it is not a minimal promoter). Accordingly, it is not a promoter (e.g. a CMV promoter, or the minimal portion thereof) capable of initiating gene transcription.

A minimal promoter consists of elements required for the initiation of transcription, and may independently initiate a low level of transcription. It may therefore be considered to comprise the minimum components sufficient for gene expression. Thus, as referred to herein, a minimal promoter is a promoter sequence which is itself (i.e. alone) capable of initiating transcription of a gene. Together with one or more additional elements (e.g. an upstream activator sequence (UAS) in eukaryotic organisms), the minimal promoter forms a promoter. Each of the elements which make up a minimal promoter (which are referred to as minimal promoter elements herein) is therefore required but is not itself (i.e. on its own, in the absence of other elements of a minimal promoter) capable of initiating transcription (i.e. is necessary but not sufficient for initiating transcription).

A minimal promoter in prokaryotic organisms is also known as a promoter core, and consists of a transcription start site and sequences capable of interacting with a σ factor (sigma factor) at the −10 and −35 positions relative to the transcription start site (Zong et al. 2017. Nature Communications 8:52). Collectively, the sequences capable of interacting with a σ factor may be known as a factor binding sites. A wide variety of a factor binding sites are known in the art, and comprise a range of different sequences. In certain embodiments, the a factor binding site at the −10 position may be for the σ⁷⁰ factor, and may a Pribnow box comprising the “TATAAT” (SEQ ID NO:46) consensus sequence. It is known in the art that only a small proportion of Pribnow box sequences comprise this consensus sequence, and many Pribnow box sequences may comprise a sequence having one or more nucleotide substitutions relative to this consensus sequence. Alternative a factor binding sites are known in the art, including σ¹⁹, σ²⁴, σ²⁸, σ³², σ³⁸ and σ⁵⁴.

A minimal promoter in eukaryotic organisms, including yeast, generally consists of a transcription start site, a TATA box, which is found 25-35 nucleotides upstream of the transcription start site and which is capable of interacting with a transcription initiation factor, and a DNA sequence that interacts with an RNA polymerase, which is found between the TATA box and the transcription start site (Redden & Alper 2015. Nature Communications 6:7810). The consensus sequence for a TATA box in Saccharomyces cerevisiae is TATA(A/T)A(A/T) (SEQ ID NO:47). It is known in the art that only a small proportion of TATA boxes comprise this consensus sequence, and many TATA boxes may comprise a sequence having one or more nucleotide substitutions relative to this consensus sequence. Alternatively, in eukaryotes an initiator element (Inr) is capable of initiation transcription on its own, i.e. in the absence of additional elements, and constitutes a different class of minimal promoter.

In certain embodiments, the fixed sequence therefore may be a sequence which ensures that translation of an mRNA product formed following transcription of a gene can take place, i.e. a translation modulating sequence. Thus, the fixed sequence may be a sequence essential or important, or beneficial, for the initiation of translation. In certain embodiments, the fixed sequence may therefore be the binding site for a protein which allows recruitment of a ribosomal subunit or a ribosome, e.g. eIF3. Alternatively the fixed sequence may itself be a binding site for a ribosome, e.g. a Shine-Dalgarno sequence, and may be situated around 7 nucleotides upstream of the translation initiation codon AUG in the corresponding mRNA, i.e. 5, 6, 7, 8, 9, 10 or 11 nucleotides upstream. In yet further embodiments, the fixed sequence may be a sequence which indicates the position for the initiation of translation, e.g. a Kozak sequence.

In further embodiments, the fixed sequence may be a sequence required for the initiation of transcription, e.g. a sequence which allows the recruitment of RNA polymerase. Thus, in certain embodiments the fixed sequence may be a TATA box, and may be situated 25-35 nucleotides upstream of the transcription start site. In other representative embodiments, however, where a fixed sequence is provided, the fixed sequence is not a sequence for the initiation of transcription (e.g. it is not a sequence which is required is for the initiation of transcription). Put another way, the fixed sequence is not a transcriptional regulatory sequence. Thus, in an embodiment, the fixed and predetermined sequence is not a predetermined promoter sequence capable of initiating transcription, or any part or element thereof. In other words, in such an embodiment the fixed and predetermined sequence is not a promoter element or promoter sequence, or more particularly it is not a known or recognised promoter element or promoter sequence. In particular, the fixed sequence may not be a promoter sequence, or a minimal promoter sequence which is itself capable of allowing transcription. Yet more particularly, the fixed sequence may not be an element of a minimal promoter i.e. a sequence which is necessary but which itself not sufficient for the initiation of transcription as described above, for example a DNA sequence that interacts with an RNA polymerase, a TATA box, a transcription initiation sequence, or a sequence capable of interacting with a σ factor, such as a Pribnow box. Thus, in certain embodiments, the fixed sequence may not be a consensus sequence for a minimal promoter element, or a variation thereof. In yet further embodiments, the fixed sequence may not be an initiator element.

Accordingly, whilst the fixed sequence may be fixed within a library of nucleic acid molecules for use in a method of identifying an element which modulates gene expression, the particular sequence which is to be fixed may be selected according to the organism and/or species for which additional elements which regulate gene expression are to be identified, based on well-known sequences available in the art.

As noted above, the stochastic sequence may alternatively or additionally comprise one or more biased random nucleotides at a given position or positions within the library of nucleic acid molecules, at which a given nucleotide is present at greater of less than the frequency (i.e. the random frequency) which would be expected, i.e. greater or less than the stochastic frequency. That is to say, for example, that based on the canonical four DNA nucleotide bases “A”, “T”, “C” and “G”, the frequency of finding any given nucleotide base at any given position within the respective nucleic acid molecules which make up the library would be expected to be approximately 25%, but that in certain embodiments, a biased random nucleotide may be present to a greater or lesser extent at a given position.

A nucleotide which occurs at a greater frequency than random at a given position may be described as biased towards, and a nucleotide which occurs at a lower frequency than random at a given position may be described as biased against. For example, where there is a bias towards a particular nucleotide, that nucleotide would be present at a particular position within the nucleic acid molecules which form the library at a frequency of greater than the stochastic frequency, and where there is a bias against a particular nucleotide, that nucleotide would be present at a particular position within the nucleic acid molecules which form the library at a frequency of less than the stochastic frequency.

Alternatively put, the library of nucleic acid molecules may be designed such that at a given position, the nucleic acids comprise a particular nucleotide at a given position at greater or less than the stochastic frequency, e.g. greater than or less than 25% of the nucleic acid molecules in the library will comprise a particular nucleotide at a given position where “A”, “T”, “C” and “G” nucleotides are used. Put another way, the probability of finding a given nucleotide at a given position within the library of nucleic acid molecules may be greater or less than the stochastic frequency.

By way of illustrative example, the stochastic sequence may be biased towards “A” and/or “T” at a given position. Thus, based on the above four canonical nucleotide bases, greater than 25% of the library of nucleic acid molecules may comprise an “A” nucleotide and/or greater than 25% of the library of nucleic acid molecules may comprise a “T” nucleotide at a given position. Alternatively put, greater than 50% of the library of nucleic acid molecules may comprise an “A” or a “T” nucleotide at a given position.

The stochastic sequence may similarly comprise a biased random sequence, i.e. a stretch of two or more contiguous random biased nucleotides as described above, thereby forming a sequence which is itself biased towards or against one or more particular nucleotides. A random biased sequence which is biased towards a given nucleotide or nucleotides may be said to be “rich” in that particular nucleotide or nucleotides.

A biased random sequence may preferably comprise 3, 4, 5, 6, 7, 8, 9, 10, 11, 12, 13, 14, 15 or more nucleotides, e.g. at least 20, 25, or 30 nucleotides. The composition of the stochastic sequence across the biased random sequence is therefore greater than or less than a stochastic amount of a given nucleotide. In certain embodiments, the entire stochastic sequence may be a biased random sequence, e.g. the entire stochastic sequence may be A/T rich.

Furthermore, the stochastic sequence may comprise two or more biased random sequences which may be the same or different, i.e. the length and/or composition of each such sequence may therefore be the same or different. In certain embodiments, the stochastic sequence may therefore comprise three, four, five or six or more random biased sequences.

In a particularly preferred embodiment, the stochastic sequence may comprise one or more biased random sequences which are A/T rich, i.e. which comprise two or more nucleotides which are biased towards A and/or T nucleotides. In a yet further preferred embodiment, the stochastic sequence may comprise two biased random sequences which are A/T rich, and in a particular embodiment said sequences may be situated at the −35 and/or −10 regions (relative to the transcription start site of a gene).

In an embodiment, the stochastic sequence does not comprise multiple repeats of a shorter stochastic sequence. In other words, the stochastic sequence does not comprise 2 or more, or 3, 4, 5 or 6 or more, repeats of a stochastic sequence. Accordingly, in an embodiment the nucleic acid molecule comprises a single copy of the stochastic sequence.

The nucleic acid molecules may comprise, in addition to the test sequence defined above (i.e. the stochastic sequence and optional fixed and pre-determined sequence) one or more sequences which are necessary for generating and/or processing the nucleic acid molecule. These may comprise, for example, one or more primer binding sites (e.g. for amplification), immobilisation sequences complementary to a nucleic acid molecule conjugated to a solid surface, cleavage sites (e.g. restriction sites for cloning), hairpin structures, and/or sequence complementary to a detection oligonucleotide optionally hybridised to a detection label (e.g. a TaqMan probe).

As defined above, the stochastic sequence (which includes any biased random sequences) and optional pre-determined or fixed sequence together represent a sequence of interest, i.e. a test sequence, and reference to the size or length of the nucleic acid molecule therefore refers to this sequence. Any additional or accessory sequences (e.g. which might be required for generating and/or processing the nucleic acid molecules) are provided in addition to this sequence, and thus reference to a nucleic acid molecule comprising at least a certain number of nucleotides does not include any such additional sequences.

Methods of preparing a library of nucleic acid molecules having stochastic sequences are known in the art, and an illustrative method of doing so is provided in the Examples below. Without wishing to be bound by theory, it is believed that altering the concentration of each nucleotide analogue added during the solid phase synthesis method described below can influence the probability of each nascent nucleic acid molecule comprising a particular nucleotide at a given position, thereby allowing a biased random nucleotide or sequence to be achieved. The preparation of nucleic acid molecules for introduction into expression vectors is described in more detail below.

In a particular embodiment, the present invention provides a method of screening test sequences for whether they comprise an element which modulates gene expression, and subsequently identifying the test sequence, thereby to identify an element which modulates gene expression.

Certain stochastic sequences provided in the methods of the present invention may comprise elements which modulate gene expression. Put another way, elements identified according to methods of the present invention are found within or comprise the stochastic sequence of a nucleic acid molecule that is provided in the methods of the present invention.

As noted above, an element which modulates gene expression as described herein may be any element which mediates, affects or alters the level or rate of transcription and/or translation of a gene. The element may therefore be any regulatory sequence which can control or alter expression of a gene.

The element may therefore be a transcriptional and/or translational control sequence, or a sequence which interacts with a transcription and/or translation modulating factor.

The element may, therefore, be a binding site for a regulatory molecule that activates or inhibits transcription. In certain embodiments, the element may be the binding site for a transcription factor. The present invention allows both eukaryotic and prokaryotic elements to be identified, and suitable host cells and/or reporter systems may be selected by the skilled person based on the organism for which new elements which modulate gene expression are desired.

Thus, the element may be a binding site for a eukaryotic transcription factor, i.e. a sequence-specific DNA-binding factor that can control the rate of transcription of a gene. Transcription factors may be either an activator or a repressor of gene expression; a repressor typically downregulates gene expression and acts by preventing RNA polymerase from forming a productive complex with the transcriptional initiation region, while activators upregulate gene expression by facilitating formation of a binding complex and can recruit RNA polymerase. The element may therefore be a binding site for any transcription factor which recruits a eukaryotic RNA polymerase enzyme, such as RNA polymerase I, RNA polymerase II or RNA polymerase Ill.

In further embodiments, the element may be a binding site for a prokaryotic transcription factor. In particular, the element may be the binding site for a prokaryotic transcription initiation factor, such as a bacterial sigma factor protein, which in turn can recruit RNA polymerase to a site upstream of a gene. Alternatively, the element may be an operator, i.e. a binding site for a repressor (e.g. a negative inducible repressor or a negative repressible repressor). A repressor binds to an operator, which is typically situated downstream of a promoter, and physically prevents procession of the RNA polymerase, thereby to prevent gene expression. In particular embodiments, and as discussed elsewhere in greater detail, the element may be the binding site for a hitherto unknown transcription factor. Thus, an element identified according to the method of the present invention may subsequently lead to the discovery and identification of new transcription factors and/or classes of transcription factors.

A transcription factor as discussed here (i.e. a transcription factor which can bind to an element, thereby to modulate gene expression) may itself be inducible or repressible in response to a particular condition or stimulus, and thus as described in greater detail elsewhere, the present invention allows conditions which can modulate gene expression to be identified.

Thus, the element may be a binding site for any regulatory molecule which may modulate gene expression, or which mediates or takes part in a reaction or process which modulates gene expression. It may be also be a sequence which itself modulates gene expression. In addition to the elements discussed above, mention may be made of promoter, enhancer and silencer sequences, or sequences which when transcribed into RNA may modulate gene expression, e.g. which provide binding sites for molecules involved in translation (e.g. a translation modulating factor), or which may affect transcript stability, or which may interact with other parts of the mRNA, for example to stabilise the transcript, or modulate the mRNA through cleavage etc. The element, when transcribed into mRNA, may affect the secondary structure of the mRNA, which may have an effect on gene expression.

In a particular embodiment, the element may be a eukaryotic or prokaryotic promoter sequence or functional portion thereof, or an element thereof. The element may be a constitutive promoter sequence, or alternatively may be an inducible promoter sequence, i.e. a promoter sequence which is capable of initiating transcription only under particular conditions and/or in response to an external stimulus.

In further embodiments, an element may be the binding site for an enzyme. In particular, the element may be the binding site for a polymerase enzyme, or may be the binding site for a ribosome (i.e. it may be a ribosome binding site (RBS)). For example it may be a Shine-Dalgarno sequence, or a binding site for a protein which allows recruitment of a ribosomal subunit or a ribosome, e.g. eIF3, or a Kozak sequence, or such like, as discussed above.

In a particular embodiment, the element may be a sequence (or more specifically the reverse complement of a sequence) which interacts with a further sequence in an mRNA transcription product, e.g. a sequence within one or more introns or within the 3′ UTR or an mRNA molecule. This interaction may, for example, affect gene splicing, and/or mRNA stability, thereby modulating gene expression. Thus, the element may be a 5′ UTR sequence, or more particularly a functional part of a 5′UTR sequence.

In an embodiment the element may be a promoter-5′ UTR sequence. In other words, it may represent a synthetic promoter-5′UTR sequence, or a combined promoter-5′UTR sequence.

It will therefore be apparent that any element which can modulate gene expression may be identified according to the methods of the present invention. Following identification of the element, however, it is preferred that the nucleic acid molecule comprising the element is sequenced, in order to determine the nucleic acid sequence of any element identified by the method of the present invention. Thus, in a preferred embodiment, the sequence of the stochastic sequence of a nucleic acid molecule identified by the method of the present invention is determined, i.e. step (e) comprises sequencing the stochastic sequence of an identified nucleic acid molecule.

Preparing a library of test constructs each comprising a nucleic acid molecule as defined above may alternatively be understood to mean introducing or inserting nucleic acid molecules into a nucleic acid construct. Nucleic acid molecules as defined above are introduced or inserted into nucleic acid constructs upstream of a reporter sequence, thereby to provide test constructs, and more particularly, a library of test constructs, wherein each said test construct comprises a nucleic acid molecule having a stochastic sequence and an optional pre-determined or fixed sequence. As described in greater detail above, the test constructs do not comprise a separate sequence upstream of the reporter sequence that can modulate expression of the reporter sequence separate or further to the nucleic acid molecule (which may include an optional fixed sequence). In particular embodiments, the test construct therefore does not comprise a separate or further transcriptional regulatory sequence, such as a promoter or minimal promoter sequence, a transcription initiation sequence, or a minimal promoter element such as a TATA box or Pribnow box (or variant thereof).

A nucleic acid molecule may be composed of deoxyribonucleic acids (DNA), ribonucleic acids (RNA) or a combination thereof, depending on the process by which a library of nucleic acid molecules is provided. However, in order for a nucleic acid molecule to be inserted into nucleic acid constructs for subsequent assessing of the function of its stochastic sequence, the nucleic acid molecule is converted into a double-stranded DNA molecule. Thus, a nucleic acid molecule may be single-stranded or double-stranded (or partially double-stranded), however, prior to insertion into a nucleic acid construct it is modified to be a double-stranded DNA molecule. Such modification may be performed in a number of ways, and provided herewith is a non-limiting selection of possible protocols therefor.

Thus, a single-stranded molecule may be prepared comprising the test sequence (i.e. the stochastic sequence and optional fixed and pre-determined sequence). It may then be manipulated in various ways to synthesise a second, complementary strand. For example, the molecule may be provided with adaptor sequences at each end of the test sequence which contain primer binding sites for amplification, e.g. by PCR. As described further below, the molecule may further comprise restriction sites, which may lie between the respective adaptor sequences, and flank the test sequence. The provision of such adaptor sequences to provide primer binding sites etc. is well known in the art, and well known systems, such as the provision of BioBrick prefix and suffix sequences, may be used.

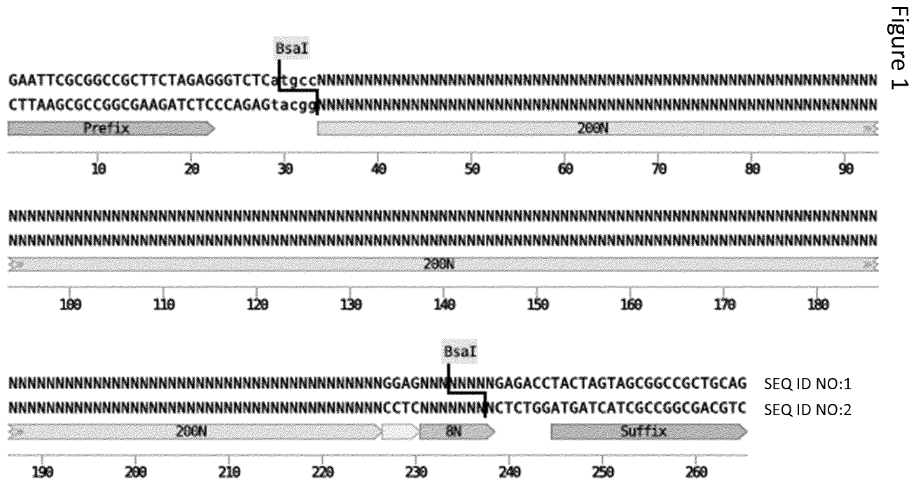

Accordingly, in one embodiment the nucleic acid molecules of the library according to the invention may be double-stranded nucleic acid molecules each comprising adaptor sequences comprising primer binding sites and restriction sites flanking each end of the test sequence. Such an arrangement is shown in FIG. 1. As can be seen from this figure, in such an embodiment, the test sequence may be flanked at each end by an adaptor sequence and a restriction site sequence and the restriction sites are located “inside” the primer binding sites in the adaptor sequences (in the sense that the restriction sites are separate sequences that lie on the inside of the primer binding site sequences, with respect to the test sequence rather than being sequences which are comprised within the primer binding sites). Accordingly, in one embodiment for preparing the molecule, a single-stranded DNA nucleic acid molecule may comprise PCR primer binding sites at its 5′ and 3′ ends. A double-stranded molecule may be generated using suitable primers in PCR. Preferably, a low number of cycles may be chosen to allow amplification of all of the nucleic acid molecules in the sample. The double-stranded molecule may be cleaved and ligated into a nucleic acid construct as described below.

In an alternative embodiment, single-stranded DNA nucleic acid molecules may be allowed to anneal to one another, and single-stranded gaps filled by a suitable DNA polymerase or Klenow fragment in the presence of nucleotides. This approach will result in double stranded DNA fragments. These may be cleaved, and the double stranded DNA fragments can then be ligated into a nucleic acid construct as described below.

In yet another embodiment, a single-stranded DNA nucleic acid molecule may form a complementary hairpin loop containing a restriction enzyme recognition site in the stem of the loop. The 3′ end of the hairpin loop can be used as a primer for DNA polymerase (in the presence of nucleotides) to synthesize the complementary strand using the 5′ portion of the nucleic acid molecule as an extension template, thereby generating a double-stranded piece of DNA. A restriction enzyme can then be used to cut adjacent to the hair pin loop, and the double-stranded nucleic acid molecule can be ligated into a vector as described below.

In a yet further embodiment, where the nucleic acid molecule is RNA, a reverse transcriptase enzyme may be used to generate a complementary strand, which may be itself be used as an extension template, thereby to generate a double-stranded DNA molecule that can be inserted into a vector as described below.

The nucleic acid molecule may be produced using a variety of different techniques. In certain embodiments, the nucleic acid molecule may be provided as a single-stranded DNA molecule. A single-stranded DNA nucleic acid molecule having a stochastic sequence may be provided using solid phase DNA synthesis, wherein a stochastic sequence may be produced using mixture of deoxyribonucleotide analogues at each stage of synthesis (i.e. for every extension step where a stochastic sequence is required. In this way, additional and/or accessory sequences (e.g. to perform any or all of the further steps outlined above to allow the nucleic acid molecule to be inserted into a nucleic acid construct) may be straightforwardly introduced. Further possible embodiments include template-free polymerase-free DNA polymerisation, which utilises a DNA ligase and a restriction endonuclease to perform DNA synthesis, or alternatively, the nucleic acid molecule may be generated using non-template polymerization of free nucleotides.

Alternatively, the nucleic acid molecule may be provided as a single-stranded RNA molecule. A single-stranded RNA nucleic acid molecule having a stochastic sequence may be provided using autocatalytic T7 RNA polymerase to generate a stochastic sequence.

Optionally, the library of nucleic acid molecules may be amplified prior to insertion into the nucleic acid constructs, and any amplification technique known in the art may be used. Amplification may be linear or exponential, as desired, where representative amplification protocols of interest include, but are not limited to: polymerase chain reaction (PCR); isothermal amplification, rolling-circle amplification (RCA), and their well-known variants, such as hyperbranched RCA, etc. Other nucleic acid amplification methods may include Loop mediated isothermal amplification (LAMP), SMart Amplification Process (SMAP), Nucleic acid sequence based amplification (NASBA), or ligase chain reaction (LCR). However, preferably amplification is performed by PCR.

A nucleic acid molecule may be inserted into a nucleic acid construct according to any convenient protocol. The double-stranded nucleic acid molecule may be inserted into a nucleic acid construct using standard recombinant DNA technology. In one embodiment, the nucleic acid molecule may comprise a restriction enzyme recognition sequence, and the nucleic acid molecule and nucleic acid construct may be cleaved by a restriction enzyme and the cleaved ends may be ligated to one-another. Cleavage by a restriction enzyme may generate blunt or sticky ends in the nucleic acid molecule and nucleic acid construct, and which may be joined by ligation. In a preferred embodiment, this may be performed by cleaving the nucleic acid molecule and nucleic acid construct with a suitable restriction enzyme or enzymes to generate mutually complementary sticky ends in the nucleic acid molecule and nucleic acid construct.

In a particularly preferred embodiment, the restriction enzyme is a type IIs restriction enzyme, i.e. an enzyme which recognises a specific sequence in double-stranded DNA but which cuts DNA at a defined distance away from the recognition site. Exemplary type IIs restriction enzymes include, for example, BbsI, BsaI, FokI, MmeI and SapI, and each recognise a different nucleic acid sequence and cut at different distances from the recognition sequence.

In certain embodiments, therefore, a nucleic acid molecule may comprise in addition to the stochastic sequence and optional pre-determined and fixed sequence, a sequence or sequences (e.g. sequences 5′ and 3′ to the stochastic sequence and optional fixed sequence) which are suitable for insertion of a nucleic acid molecule into a nucleic acid construct. In a preferred embodiment, this may be a restriction enzyme recognition sequence, or in particular, a type IIs restriction enzyme recognition sequence. The nucleic acid construct also preferably comprises sequences ‘compatible’ with the above sequence or sequences, in order to allow the insertion of a nucleic acid molecule.

Preparing a library of test constructs may be performed by separately inserting separate members of a library of nucleic acid molecules into a nucleic acid construct according to any of the methods outlined above. However, in a preferred embodiment, the library of nucleic acid molecules may be pooled (i.e. combined) prior to or during step (b), before insertion of the nucleic acid molecules into the nucleic acid constructs. Thus, in certain embodiments, the pooled library of nucleic acid molecules may be inserted into the nucleic acid constructs by treating the pooled library of nucleic acid molecules in such a way to prepare them for insertion into nucleic acid constructs as outlined above, e.g. by enzymatic cleavage, and inserting nucleic acid molecules into nucleic acid constructs. Thus, different nucleic acid molecules may independently be inserted into respective nucleic acid constructs in a single reaction vessel or volume, and a library of test constructs, each comprising a nucleic acid molecule, may thereby be generated. As described in greater detail above, however, it will be understood that in certain embodiments only a selection of the available nucleic acid molecules may be introduced into nucleic acid constructs in such a method, due to the stochastic nature of the molecular steps required to insert a nucleic acid molecule into a nucleic acid construct. The library of test constructs may therefore comprise only a sub-set of the nucleic acid molecules provided in step (a) of the methods of the present invention. Nevertheless, a library of nucleic acid molecules prepared in this way comprises a representative sample of the stochastic sequences provided in the nucleic acid molecules of step (a).

In certain representative embodiments, the library of test constructs generated in step (b) may optionally be amplified between steps (b) and (c) to increase the copy number of each test construct in the library. Such a step may be performed to improve the efficiency with which the test constructs are inserted into host cells in step (c).

Said amplification may be performed using any of the molecular amplification techniques outlined above with respect to amplifying the nucleic acid molecules. However, in a representative embodiment, said optional amplification step may comprise introducing the library of test constructs into intermediate cells (i.e. intermediate with respect to the host cells in which expression of the reporter sequence is eventually assessed), and said cells may be cultured to allow amplification of the library of test constructs.

In certain embodiments, test constructs amplified in this way may be isolated from said intermediate cells according to any convenient protocol (e.g. a Qiagen miniprep nucleic acid isolation protocol), and the thus isolated test constructs may be introduced into host cells in step (c).

In one embodiment, following the introduction of the library of test constructs to the intermediate cells, the intermediate cells may be plated on a solid growth medium, e.g. a growth medium suitable for the growth of microbial cells, such as a 2 TY agar medium and the cells may be incubated. Individual cells or colonies comprising a test construct may be selected, and a test construct isolated therefrom and introduced into a host cell (which process may be separately performed in a multiplexed manner (i.e. to allow a plurality of test constructs to be isolated from a plurality of intermediate cells in order to allow a plurality of test constructs to be introduced into host cells). In an alternative embodiment, however, intermediate cells prepared in this way may be pooled and test constructs isolated therefrom, and the resulting library of test constructs may be used in step (c) of the present invention.

Intermediate cells for use in such an optional amplification step may preferably be competent cells, and may include chemically competent cells or electrocompetent cells. In particular embodiments, DH5a E. coli cells or turbo competent E. coli C2984 may be used for such a step.

In yet further embodiments, the library of test constructs (optionally having been amplified as outlined above) may be introduced into an intermediate cell capable of initiating conjugal transfer of a test construct into a host cell. Introduction of a library of test constructs into host cells may therefore comprise the introduction of a library of test constructs into an intermediate cell capable of initiating conjugal transfer of the test constructs into host cells, and introducing the test constructs into host cells via conjugal transfer. Preferred cells for such a step may be competent cells, e.g. chemically or electrically competent cells. In one embodiment, the cells may be transform competent E. coli S17.1 cells

Thus, the insertion of the library of nucleic acid molecules into respective nucleic acid constructs allows a library of test constructs, ideally each comprising a different nucleic acid sequence, to be provided. It will therefore be understood that each test construct thus formed will therefore comprise a stochastic sequence and optionally a pre-determined and fixed sequence, as per its respective nucleic acid molecule. Introduction of the library of test constructs into host cells may similarly be performed by separately introducing members of the library of test constructs (or a part or portion thereof, as discussed above) into host cells by any standard technique, e.g. as described herein. However, preferably, a library of test constructs may be pooled (i.e. combined) prior to or during step (c), before introduction of the test construct into the host cells. Thus, in certain embodiments, the pooled library of test constructs may be introduced into the host cells by contacting a population of host cells with the pooled library of test constructs, and subjecting the population of host cells to necessary conditions to allow the introduction of the test constructs thereto. Thus, test constructs comprising different nucleic acid molecules may be introduced into host cells in a single reaction vessel or volume, and a library of test constructs, each comprising a nucleic acid molecule, may thereby be introduced into a population of host cells, thereby to generate a host cell library. In a preferred embodiment, each of the host cells comprises, or is intended to comprise, a different test construct, i.e. comprising a different nucleic acid molecule.

A test construct may be introduced into a host cell by any convenient method known in the art, and the method selected may depend on the nature and identity of the host cell and/or the test construct.

A test construct may be introduced into a prokaryotic host cell by transformation, i.e. direct uptake through the cell membrane. Transformation is a horizontal gene transfer method which allows genetic material to be introduced to receptive cells (e.g. competent bacterial cells), and may be performed e.g. by directly contacting a host cell with a test construct, or by electroporation. Bacteriophage may alternatively be used to introduce the test construct into a prokaryotic host cell.

The test construct may be introduced into certain eukaryotic cells, e.g. yeast and plants, by transformation. Yeast may be transformed, for example, by treating cells with enzymes to degrade their cell walls, exposing cells to alkali metal cations (e.g. lithium or caesium), electroporation or enzymatic digestion. Plant cells may also be transformed e.g. using Agrobacterium-mediated transformation, microprojectile bombardment using particles coated with test construct, or electroporation. Viral transduction may alternatively be used.

Test constructs may alternatively be introduced into animal cells by transfection, e.g. using a standard calcium phosphate transfection, electroporation, sonoporation, cell squeezing or lipofection using a cationic lipid (e.g. lipofectamine or similar reagent) to generate liposomes for transfection. Viral transduction may alternatively be used.

The test construct may be any construct suitable for introduction into a host cell, and may be selected to comprise one or more suitable markers (e.g. genetic markers), origins of replication and/or genome incorporation sequences to allow the vector to be maintained in a population of host cells. Thus, the identity and nature of these components of the test construct may be selected by the skilled person based on the identity of the host cell which is to be used in the methods of the present invention.

The test construct may therefore be a vector which carries the nucleic acid and/or which enables or facilitates its transfer or introduction into a host cell. In other words the test construct may be any construct which carries or allows the transfer or introduction of the nucleic acid molecule (with the reporter sequence) into a host cell. In certain embodiments it may be an expression vector, or it may be, or may comprise an expression cassette. It may thus be a plasmid or a virus designed for gene expression in host cells, or a construct which can be stably integrated into the genome of a host cell, either in a specific or non-specific manner. Thus, the test construct may be stably inserted into a host cell, e.g. by transformation or transfection (i.e. it may be incorporated into a host cell's genome), or transiently inserted into a host cell (i.e. such that it is maintained episomally in a host cell, as a separate and independent construct that is not inserted into a host cell's genome. The test construct may therefore comprise suitable sequences (e.g. for insertion or episomal maintenance) to allow it to perform the required functions as described above.

As described in greater detail above, the skilled person would understand that the various techniques discussed above are not 100% efficient at introducing genetic material into a host cell, and furthermore that only a sub-set of the library of test construct s may be successfully introduced into host cells. Nevertheless, host cells comprising a representative sample of the nucleic acid library of step (a) may be prepared.

The test construct comprises a reporter sequence, which is operatively linked to the nucleic acid molecule introduced into a test construct. Thus, the nucleic acid construct (into which the nucleic acid molecule is introduced) comprises a reporter sequence, and the nucleic acid molecule becomes operably linked to the reporter sequence in the test construct. The nucleic acid molecule is introduced into the nucleic acid construct upstream (i.e. in the 5′ direction) of the reporter sequence to form the test construct, and as noted above, the test construct does not comprise any further separate sequence that can modulate expression of the reporter sequence. Thus, expression of the reporter sequence is entirely under the control of the nucleic acid sequence that is introduced into the nucleic acid construct to generate a test construct, and assessment of the level of expression of the reporter sequence allows the identification of any nucleic acid molecule (and thus element) which can modulate the level of gene expression. The reporter sequence comprises a reporter gene and at its simplest it may be a reporter gene. Assessment of the expression of the reporter sequence therefore comprises assessment of the expression of the reporter gene. The reporter gene may be any gene which can be detected in order to assess the level of expression that is driven by the nucleic acid molecule.

The test construct does not comprise a further, separate, sequence upstream of the reporter sequence that can modulate expression of the reporter sequence. Thus, the test construct does not contain any other sequence, beyond the introduced nucleic acid molecule (that is beyond the stochastic sequence or pre-determined and fixed sequence within or adjacent thereto), that may act as regulatory, or expression control sequence to direct or control, or in any way modulate, the expression of the reporter sequence.

In particular, the test construct does not include, operably linked to the reporter sequence, any sequence further to or separate from the nucleic acid molecule or pre-determined and fixed sequence within or adjacent thereto which is known or pre-determined to be an expression control sequence, or to modulate gene expression. In particular, the test construct does not include a pre-determined, or known, promoter sequence. More particularly it does not include a sequence which is a pre-determined minimal promoter sequence, or a minimal promoter element as described above.

It will be understood, however, that in certain embodiments, the test construct or the nucleic acid construct may comprise one or more additional genes or operons which are not operably linked to the reporter sequence (or the introduced nucleic acid molecule), which may independently be under the control of a separate sequence (e.g. a separate promoter sequence) capable of initiation gene expression.

The expression of the reporter gene may be assessed by detecting a transcription product (e.g. an RNA product such as an immature mRNA molecule or an mRNA molecule) of the reporter gene. Put another way, a transcription product of the reporter gene may be assessed in order to determine expression of the reporter gene. A transcription product may be detected in a variety of ways, for example a G-less cassette transcription assay, a run-off transcription assay, an RNAse protection assay, RT-PCR, gene microarray, in situ hybridisation, M2S tagging, Northern blot, or next-generation sequencing (RNA-Seq).

Alternatively or additionally, the expression of the reporter gene may be assessed by detecting a protein gene product of the reporter gene. This may be done directly, i.e. by assessing the level of the protein by suitable biochemical means e.g. by ELISA, Western blot, gel electrophoresis, immunostaining, chromatography, or mass spectrometry (including LC/MS).

Alternatively, in certain embodiments, the protein may give rise to a detectable phenotype in a host cell, which may be detected in order to assess the expression of the reporter gene. Thus, in certain embodiments, the reporter gene may be a selection marker, e.g. a positive selection marker such as an antibiotic resistance gene or an orotidinie-5′ phosphate decarboxylase gene. Assessment of expression of the reporter gene may therefore be performed by incubating host cells comprising a nucleic acid molecule as defined above in the presence of a suitable selection reagent, in order to screen for host cells which express the selection marker.

In alternative embodiments, the reporter gene may be a screenable marker, such that host cells in which the reporter gene is expressed appear different due to the protein being coloured and/or fluorescent. Suitable such reporter genes therefore include genes for a coloured or fluorescent protein (e.g. GFP, YFP, RFP, mCherry or luciferase), or a protein which causes a colour change in a host cell following enzymatic breakdown of a precursor compound, e.g. β-glucurinidase, or IacZ. Thus, in preferred embodiments, expression may be assessed by detecting a colour change or fluorescence in a host cell following a period of incubation. In a particularly preferred embodiment, the reporter gene encodes a fluorescent protein and detection is performed by flow cytometry.

In certain embodiments, the reporter gene may be provided as part of a reporter system construct. In such embodiments, the reporter gene as defined above may be provided in the same operon as, or in frame with, one or more further genes, e.g. for which is it desired to identify an element which modulates gene expression. Thus the reporter gene may be translationally coupled to the further gene (e.g. the desired, target or test gene). In this way, assessing the expression of the reporter gene may allow an element which modulates activity of that gene to be identified. Such a method may be of particular interest where it is desired to identify and element which modulates gene expression by interacting with the RNA transcript of said gene, thereby affecting its cleavage, processing and/or stability, and thus modulating the expression of said gene. The further gene may therefore be e.g. a genomic DNA sequence, i.e. comprising intronic and 3′ DNA sequences which are not translated, and which are modified and/or removed during processing of immature mRNA.

Identifying an element which modulates gene expression comprises identifying a nucleic acid molecule, and thus a stochastic sequence therein, which effects a particular or desired level of expression of the reporter gene, i.e. a level of expression of the reporter gene that is of interest. In other words, a particular or desired level of expression of that gene may be indicative of an element that modulates gene expression. Identifying an element which modulates gene expression may therefore comprise measuring the level of expression of the reporter gene in a host cell, and wherein said host cell has a particular or desired level of gene expression (i.e. a level that is of interest), that cell may be selected, in order that the nucleic acid molecule therein may be identified. Determining or identifying a level of gene expression is defined broadly herein to include qualitative, quantitative and semi-quantitative assessments, and includes detecting the presence or absence of expression. Thus a level of expression includes any level of expression, howsoever determined, and includes simply that expression is detected, or that the absence of expression is detected, as well as the amount or rate of expression, e.g. the amount of transcript and/or protein product, or the rate at which it is produced.

It may be desirable that the level of expression of the reporter gene is high, e.g. in order to identify an element which positively modulates gene expression. Alternatively, it may be desirable that the level of expression of the reporter gene is low, e.g. in order to identify an element which negatively modulates gene expression. In certain embodiments, the level of expression of the reporter gene may be a threshold level of expression, such that any level of expression that falls beyond that level may be an expression level (i.e. above or below a given threshold) that is of interest. Thus, any host cell having a level of expression of the reporter gene beyond that level (i.e. above or below that threshold as desired) may be said to have a level of expression that is of interest.

Identification of an element may comprise determining the sequence of the stochastic sequence and optional pre-determined and fixed sequence. In certain embodiments, an element may be identified following the isolation of nucleic acid molecules from two or more host cells.

Optionally, nucleic acid molecules from a number of host cells having a level of expression of the reporter gene that is of interest may be identified. Following introduction of the test construct into host cells, the host cells may preferentially be incubated for a period of time to allow expression of the reporter gene to take place. Incubation may take place in or on any suitable growth media, including both liquid and solid growth media, which may be selected based on the nature and identify of the host cells and/or the reporter gene used to assess gene expression.

Incubation may take place for a period of time sufficient to allow expression of the reporter sequence to take place (i.e. so that the level of expression may be assessed). Incubation thus may take place for at least 10 minutes, 20 minutes, 30 minutes, 40 minutes or 50 minutes, or at least 1 hour, more preferably 2, 3, 4, 5 or 6 hours, or at least overnight e.g. at least 8, 12, 16, 20 or 24 hours. Longer periods of incubation may also be performed, e.g. at least 2, 3, 4, 5, 6, or 7 days, in order to allow expression of the reporter gene. During incubation, the growth medium of the cells may be replaced and/or additional nutrients added to the growth medium to maintain the host cells.

Incubation may take place under a wide range of possible conditions, and conditions which may be selected for incubation include a particular temperature, pH, oxygen saturation, concentration of one or more salts, or exposure to light of one or more wavelengths or a spectrum of wavelengths (or incubation in darkness). Furthermore, the growth medium for incubation may be supplemented with or deficient in (i.e. incubation may take place in the presence or absence of) one or more molecules or factors, e.g. chemical factors (chemicals). In certain embodiments, therefore, the growth medium may be supplemented with one or more chemicals, including a sugar, amino acid, peptide, protein, antibiotic, putative regulatory compound or a test candidate compound.

A number of promoter-5′ UTR sequences were identified using the methods of the present invention in a range of microorganisms, as outlined in the Examples below. A summary of the promoter-5′UTR sequences identified is provided in Tables 9-15.

In a further aspect, the present invention provides a vector comprising any one of SEQ ID NOs:77-111. In particular, the vector may be an expression vector. In an embodiment the said promoter-5′UTR sequence may optionally be operably linked to a heterologous gene, such that the expression of the gene is at least partly under the control of said sequence. In an alternative embodiment, the vector comprises the promoter-5′ UTR sequence and a cloning or insertion site, such that a coding sequence (i.e. a nucleotide sequence encoding a desired product, which may be a polypeptide or RNA product) may be introduced into the site and expressed under the control of the promoter-5′ UTR sequence.

In one embodiment of the present invention, it is possible to investigate whether an element modulates gene expression under particular conditions. In other words, it may be desirable to select a particular condition as described above (or for that matter of combination of conditions, e.g. a particular temperature, pH and chemical), and investigate whether an element which modulates gene expression under that particular condition may be identified. In other words, an element which modulates gene expression under a particular condition, i.e. a test condition, is provided by the present invention, and any of the conditions described above may be a test condition according to this aspect of the present invention.

The present invention therefore provides a method for identifying an element which modulates gene expression under a test condition, wherein assessing step (d) comprises:

(i) incubating the host cells under a test condition; and

(ii) assessing expression of the reporter gene in the host cells;

wherein the nucleic acid molecule identified in step (e) is an element which modulates gene expression under a test condition.

It will be appreciated, therefore, that in such an embodiment the method may be used to identify regulatable promoters which are inducible or repressible under certain conditions, e.g. in the presence of an inducer or repressor molecule, and/or other elements which modulate gene expression in response to an external or internal stimulus, e.g. an inducer or repressor etc. Conversely, by incubating the host cells under regular or standard conditions, promoters and/or other elements that mediated constitutive expression may be identified.

The present invention also allows particular growth conditions which might modulate gene expression to be identified. More specifically, the present invention allows the identification of conditions and compounds or substances which have an effect on gene expression to be identified. In particular such methods involve the incubation of host cells under particular conditions, or put another way, the introduction of particular conditions to host cells before or during incubation, such that the host cells are incubated in the presence of that condition. A condition or conditions (i.e. combinations of different conditions as outlined above) can therefore be investigated for an ability to modulate gene expression. A condition or conditions investigated in this way may be considered to be a ‘test’ condition.

Thus, in one embodiment of the methods described above, it may be possible to identify conditions which can modulate gene expression. In other words, it may be possible to investigate whether a particular condition, i.e. a test condition, modulates gene expression by determining or identifying that a nucleic acid molecule is able to expresses the reporter gene in the host cell under the test condition. Such a method may further comprise identifying the nucleic acid molecule which expresses the reporter gene in the host cell under the test condition.

The present invention therefore provides a method of identifying a condition which modulates gene expression, wherein step (d) comprises:
(i) incubating the library of host cells under a test condition; and
(ii) assessing expression of the reporter sequence in host cells from the library of host cells;

wherein step (e) comprises identifying a nucleic acid molecule of the test construct from a host cell which expresses the reporter sequence under the test condition, thereby to identify whether the test condition modulates gene expression.

It may be possible for a test condition which modulates gene expression to be identified without also requiring the identification of an element which modulates gene expression. Therefore, in an additional aspect, the present invention provides a method of identifying a test condition which modulates gene expression, said method comprising:
(a) providing a library of at least 1×10⁶ nucleic acid molecules, wherein each nucleic acid molecule comprises a stochastic sequence of at least 50 nucleotides, optionally wherein each nucleic acid molecule also comprises an identical pre-determined and fixed sequence either adjacent or within the stochastic sequence;
(b) introducing nucleic acid molecules from said nucleic acid library of (a) into nucleic acid constructs to prepare a library of test constructs, wherein each test construct comprises a nucleic acid molecule of part (a) upstream of a reporter sequence, and wherein each test construct does not comprise a further separate sequence upstream of the reporter sequence that can modulate expression of the reporter sequence;
(c) introducing test constructs from said test construct library of (b) into host cells to prepare a library of host cells, wherein host cells from the host cell library comprise a test construct; and
(d) (i) incubating the library of host cells under a test condition; and
(ii) assessing expression of the reporter sequence in host cells from the library of host cells;

and wherein said method further comprises identifying whether the test condition modulates gene expression.

Methods disclosed herein which assess the ability of a test condition to modulate gene expression may optionally be performed by assessing expression of the reporter sequence in the presence and absence of a test condition, in order to assess the difference in gene expression under both conditions.

Thus, in certain embodiments, assessing expression of the reporter sequence in a host cell in order to assess the ability of a test condition to modulate gene expression cell may comprise providing a clonal population of a host cell (i.e. allowing a host cell to multiply to create a population of cells comprising the same nucleic acid molecule), incubating a first portion of said population under said test condition whilst incubating a second portion of said population under control conditions (i.e. in the absence of said test condition) and assessing expression of the reporter sequence in each portion, whereby any difference in the level of expression in each portion indicates that that test condition modulates gene expression. Assessing the level of expression of the reporter sequence in this way for two or more host cells may also be performed, i.e. a separate clonal population of each cell may be generated and expression assessed for each host cell.

In alternative embodiments, following the introduction of the library of test constructs into host cells, the methods may comprise separating the host cells (i.e. the plurality of host cells, each comprising a different test construct from the library of test constructs) into two portions, and incubating a first portion of the host cells under said test condition whilst incubating a second portion of the host cells under control conditions (i.e. in the absence of the test condition as defined above). Assessing the level of expression of the reporter sequence in host cells from each portion (i.e. the portion of host cells incubated in the presence and absence of the test condition) may allow the identification of a test condition which modulates gene expression.

As a yet further alternative embodiment, a test condition may be introduced during the course of incubating the host cells, and the level of expression of the reporter sequence may be assessed before and after the introduction of the test condition. In other words, incubation may comprise a first incubation period in the absence of the test condition, followed by a second incubation period in the presence of the test condition, and expression may be assessed at one or more points during one or both periods.

It is therefore possible according to any one of these embodiments to assess whether a test condition can modulate gene expression. As noted above, this may optionally be accompanied by identifying a nucleic acid molecule of the test construct from a host cell having a level of expression of the reporter construct that is of interest, thereby to identify an element which modulates gene expression.

The host cell may be any organism for which it is possible to introduce a test construct and assess a level of gene expression, and most preferably is the particular organism or species for which it is desired to identify further elements and/or test conditions which modulate gene expression. In alternative embodiments, however, it may be possible to use a particular host cell in the methods of the present invention, whereas it is desired to identify elements and/or test conditions which modulate gene expression in a different organism. In such embodiments, it may be preferable to select a host cell from the same phylogenetic domain, kingdom, phylum, class, order, family, genus or species as the organism for which elements and/or test conditions are sought. Without wishing to be bound by theory, it is believed that any elements or test conditions identified by the methods of the present invention would be more directly relevant to a more closely-related organism than a more distantly-related organism to a host cell, and optimally the host cell is therefore from the same organism as the organism for which elements and/or test conditions are sought. Furthermore, where alternative strains of or tissues from an organism are available, it would be most desirable where possible to select host cells of the same strains or tissues for performing the methods of the present invention.

Broad-host range sequences may be provided for various parameters of the present invention. For example, a pre-determined and fixed sequence which is compatible with different host cells from more than one species, genus, family, order, class, phylum, or kingdom may be provided in nucleic acid molecules from the nucleic acid library of the present invention, or a nucleic acid construct which is compatible with different host cells as described above may be provided. In this way, the same nucleic acid library or test construct library prepared according to the methods described above may be tested or screened in different host cells. In a particular embodiment, a broad-range Shine-Delgarno sequence may be provided, i.e. which allows ribosome binding in a variety of different host cells as described above.

The host cell may therefore be from a prokaryotic or eukaryotic organism or species. Preferably, however, the host cell is a cell that may be manipulated, grown and/or culture in vitro, and thus is preferably a microorganism (prokaryotic or eukaryotic) or is a cell line derived from a eukaryotic organism.

In one embodiment, the cell is a prokaryotic cell, and may be any species or bacteria or Archaea. Accordingly, both Gram negative and Gram positive, Gram-indeterminate or Gram-non-responsive bacterial species may be selected. Particularly, genera of bacteria include Staphylococcus (including Coagulase-negative Staphylococcus), Clostridium, Salmonella, Pseudomonas, Propionibacterium, Bacillus, Lactobacillus, Legionella, Mycobacterium, Micrococcus, Fusobacterium, Moraxella, Proteus, Escherichia, Klebsiella, Acinetobacter, Burkholderia, Entercoccus, Enterobacter, Citrobacter, Haemophilus, Neisseria, Serratia, Streptococcus (including Alpha-hemolytic and Beta-hemolytic Streptococci), Bacteroides, Yersinia, and Stenotrophomas, and indeed any other enteric or coliform bacteria. Beta-hemolytic Streptococci would include Group A, Group B, Group C, Group D, Group E, Group F, Group G and Group H Streptococci, Bacteroidetes, Cyanobacteria, and Chlorobi, alpha, beta, gamma, delta and epsilon protobacteria.

Non-limiting examples of Gram-positive bacteria include Staphylococcus aureus, Staphylococcus haemolyticus, Staphylococcus epidermidis, Staphylococcus saprophyticus, Staphylococcus lugdunensis, Staphylococcus schleiferei, Staphylococcus caprae, Staphylococcus pneumoniae, Staphylococcus agalactiae, Staphylococcus pyogenes, Staphylococcus salivarius, Staphylococcus sanguinis, Staphylococcus anginosus, Streptococcus pneumoniae, Streptococcus pyogenes, Streptococcus mitis, Streptococcus agalactiae, Streptococcus anginosus, Streptococcus equinus, Streptococcus bovis, Clostridium perfringens, Enterococcus faecalis, and Enterococcus faecium, Deinococcus radiodurans, Corynebacterium glutamicum, Bacillus subtilis. Non-limiting examples of Gram-negative bacteria include Escherichia coli, Salmonella bongori, Salmonella enterica, Citrobacter koseri, Citrobacter freundii, Klebsiella pneumonia, Klebsiella oxytoca, Pseudomonas aeruginosa, Pseudomonas putida, Haemophilus influenzae, Neisseria meningitidis, Enterobacter cloacae, Enterobacter aerogenes, Serratia marcescens, Stenotrophomonas maltophilia, Morganella morganii, Bacteroides fragilis, Acinetobacter baumannii and Proteus mirabilis, Synechococcus sp. PCC 7002, Synechocystis sp. PCC 6803, Chlorobaculum tepidum, Rhodopseudomonas palustris, Rhodobacter capsulatus, Rhodobacter sphaeroides.

Particularly preferred bacterial species are Escherichia coli and Pseudomonas putida, in particular the D5a Escherichia coli strain, and the KT2440 Pseudomonas putida strain.

Host cells may alternatively be eukaryotic, and thus may include fungal, algal, plant or animal cells.

Relevant fungi may include yeasts, particularly of the genus Candida, and fungi in the genera Aspergillus, Fusarium, Penicilium, Pneumocystis, Cryptococcus, Coccidiodes, Malassezia, Trichosporon, Acremonium, Rhizopus, Mucor, Saccharomyces (including Saccharomyces cerevisiae), Schizosaccharomyces (including Schizosaccharomyces pombe) and Absidia. The host cell may be Candida or Aspergillus, and non-limiting examples of fungi include Aspergillus fumigatus, Candida albicans, Candida tropicalis, Candida glabrata, Candida dubliensis, Candida parapsilosis, and Candida krusei.

Plant cells may be any plant cell line derived from a suitable plant species, including Arabidopsis thaliana, Glycine max (soybean), Zea mays (maize), Oryza (including Oryza sativa (Asian rice) or Oryza glaberrima (African rice), Nicotiana species, Solanaceae (including Solanum lycopersicum (tomato). Algae species, including Chlamydomonas reinhardtii, Chlorella vulgaris, Nannochloropsis oceanica and Nannochloropsis gaditana may be also be suitable.

Animal cells may be derived from any suitable primary or established cell line, and may accordingly be derived from any organism. Cell lines are known from Caenorhabditis elegans (nematode worm) and Drosophila melanogaster (e.g. S2 cells) and host cells from either organism may be used in the methods of the present invention. Similarly, a wide range of cell lines are known for mice, rats or primates (including humans), and these may be used in the methods of the present invention. Cell lines including HeLa cells and any of the various HEK 293 cell lines may preferably be used.

As noted above, various parameters of the methods of the present invention may be selected based on the nature and identify of the host cell which is used in these methods, and the selection of suitable parameters based on which host cell is selected for these methods is within the judgment of the skilled person. Non-limiting examples which illustrate how such parameters may be selected in order to allow the methods of the present invention to successfully be performed are provided in the Examples below.

The invention may be better understood from the Figures, in which:

FIG. 1 shows a nucleic acid molecule comprising a 200 nt stochastic sequence flanked at its 5′ and 3′ ends by primer binding sites and restriction enzyme recognition (BsaI) sequences, and comprising an E. coli Shine-Dalgarno sequence 8 nt upstream of the position at which translation of the reporter sequence would be initiated once the nucleic acid molecule is introduced into a nucleic acid construct.

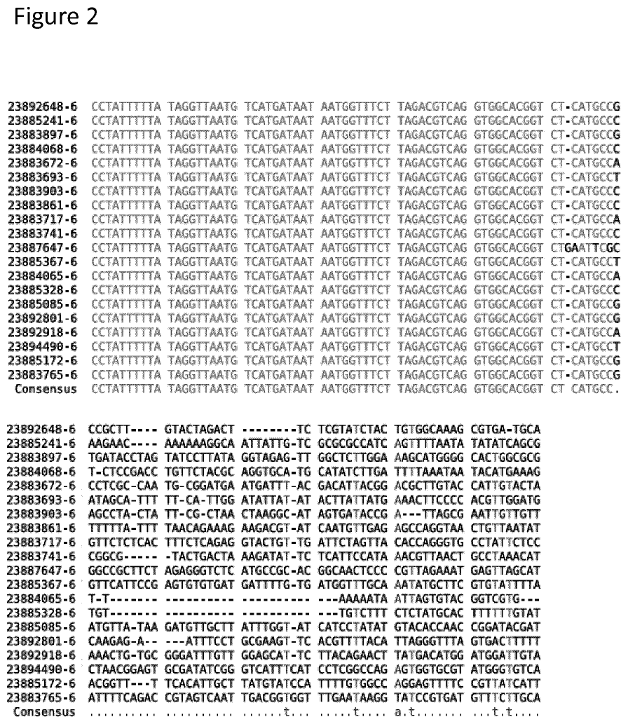

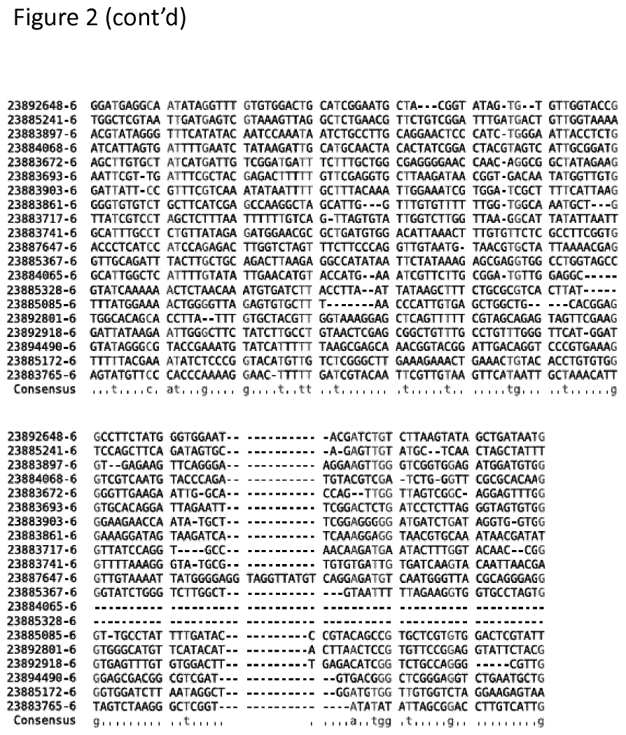

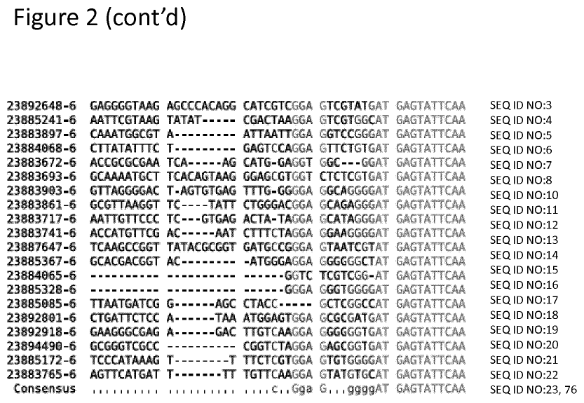

FIG. 2 shows an alignment of the sequences of 20 nucleic acid molecules identified through methods described herein, and shows that stochastic sequences are correctly provided in the test constructs and are able to drive gene expression, and that consensus sequences shared by multiple nucleic acid molecules may be identified.

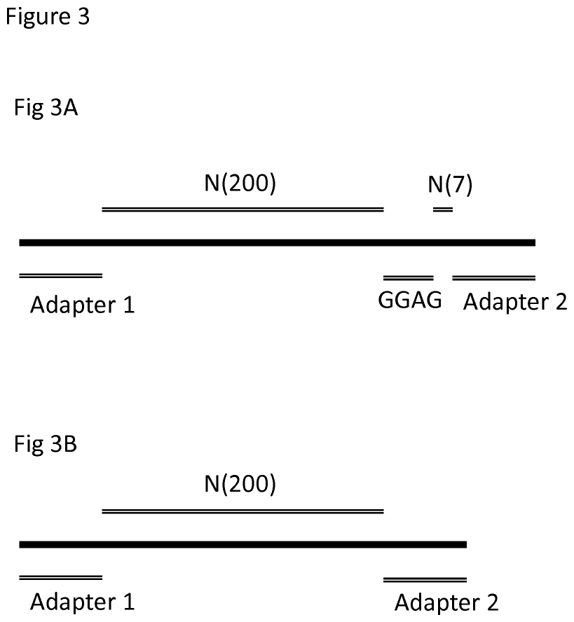

FIG. 3 shows the structural overview of synthetic DNA constructs. A, constructs including fixed Shine-Dalgarno sequence (GGAG (SEQ ID NO:45); B, only the random sequence with no fixed sequence.

### EXAMPLES

### Example 1—Beta Lactamase Reporter Sequence

A library of single-stranded nucleic acid molecules were generated by solid phase synthesis, each comprising a 200 nucleotide stochastic sequence and a 4 nucleotide pre-determined and fixed Shine-Dalgarno sequence for E. coli within the stochastic sequence. Each single-stranded nucleic acid molecule comprised primer binding sites at its 5′ and 3′ ends to allow PCR amplification, and also included BsaI restriction sites (see FIG. 1) to allow cloning into suitable vectors, e.g. a test construct containing a beta lactamase reporter sequence. The sequence of the nucleic acid molecule including primer binding sites and BSAI restriction sites is provided in SEQ ID NO:1. The reverse complement sequence is provided in SEQ ID NO:2. Following PCR amplification, double-stranded nucleic acid molecules were cloned into nucleic acid constructs to provide a library of test constructs which could be screened to assess the expression of the reporter sequence. The library of test constructs were introduced into E. coli DH5α host cells, and gene expression was assessed by plating cells on plates comprising different concentrations of ampicillin.

Twenty colonies were randomly picked and were subjected to DNA sequencing using the Bla-Rev primer (SEQ ID NO:30), leading to the promoter-5′ UTR sequences listed in FIG. 2, (performed at GATC Biotech, Germany). As expected, the sequencing results indicate the presence of different stochastic sequences in the stretch of DNA preceding the coding sequence for the beta lactamase gene. None of the sequences are identical, indicating that the screen for stochastic sequences was successful. The sequences are set forth in SEQ ID NOs:3-22.

TABLE 1

| Promoter | |
|---|---|
| sequence | SEQ ID NO: |
| 23892648-6 | 3 |
| 23885241-6 | 4 |
| 23885172-6 | 5 |
| 23883897-6 | 6 |
| 23884068-6 | 7 |
| 23883672-6 | 8 |
| 23883765-6 | 9 |
| 23883741-6 | 10 |
| 23885367-6 | 11 |
| 23883717-6 | 12 |
| 23883861-6 | 13 |
| 23883693-6 | 14 |
| 23885085-6 | 15 |
| 23892801-6 | 16 |
| 23883903-6 | 17 |
| 23887647-6 | 18 |
| 23894490-6 | 19 |
| 23884065-6 | 20 |
| 23892918-6 | 21 |
| 23885328-6 | 22 |
| Consensus | 23, 76 |

Multiple alignments of the sequenced stochastic sequences allowed consensus sequences to be identified, which correlated with high levels of expression of the reporter sequence. A portion of a consensus sequence is set forth in SEQ ID NOs:23 and 76 (representing the consensus sequences at the 5′ and the 3′ ends of the nucleic acid molecule, respectively). A Shine Dalgarno ribosome binding site (GGAG—SEQ ID NO:45) is also identifiable in the consensus sequence).

### Example 2—mCherry Reporter Sequence

The same library of nucleic acid molecules as was used in Example 1 was prepared, and nucleic acids form the nucleic acid library were cloned into a nucleic acid construct comprising a mCherry reporter sequence and transformed into E. coli cells as above. Transformed cells were screened by plating cells of plates comprising kanamycin. Following overnight incubation, 86 colonies were randomly picked and grown in 96 well plates containing a suitable growth medium. Expression levels of mCherry (red fluorescence protein) in overnight-grown cultures were assessed by fluorometric assay.

The ability to identify a factor which modulates gene expression in a further bacterial species (Pseudomonas putida KT2440) was also assessed. A library of test constructs was prepared as above and introduced into P. putida KT2440 cells, which were screened for the presence of the test construct by plating the cells on plates comprising kanamycin as above. A broad-host range replicon was used, allowing the same nucleic acid library to be screened in multiple hosts. Expression levels of mCherry in overnight-grown cultures were assessed by the same fluorometric assay, leading to identification of novel “expression-mediating StoNuSeqs” in this host.

### Materials and Methods

### Synthesis of Single-Stranded StoNuSeq (Stochastic Nucleotide Sequence)

The 265 nucleotide long stochastic nucleic acid sequence (StoNuSeq) synthetic DNA was ordered from IDT (Belgium) as a four nmole Ultramer DNA Oligo (i.e. single-stranded).

Generation of Double-Stranded StoNuSeq (ds-StoNuSeq)

The 265 nt long single-stranded StoNuSeq (ss-StoNuSeq) harbours the BioBrick prefix and suffix on its both ends as adaptors, 5′ and 3′ ends, respectively (FIG. 1). The ds-StoNuSeq was generated by using the primers BB-Prefix-Fwd (SEQ ID NO:24) and BB-Suffix-Rev (SEQ ID NO:25) in 10 cycles of PCR. The low number of cycles was chosen to prevent the reduction of complexity of the initial ss-StoNuSeq during PCR amplification.

### PCR Parameters

98° C. for 30 seconds
(98° for 10 seconds, 60° C. for 20 seconds, 72° C. for 20 seconds)×10
Construction of Bsa1-Free pUC19

The only occurring Bsa1 sequence recognition site within the beta-lactamase gene in pUC19 was eliminated by altering the sequence from GGTCTC to GGTCCC without affecting the amino acid sequence. For this the pUC19 vector was amplified using the primer pair Bsa1-Rev (SEQ ID NO:26)/Bsa1-Fwd (SEQ ID NO:27), with the following PCR parameters:

98° C. for 30 seconds
(98° C. for 10 seconds, 62° C. for 20, 72° C. for 80 seconds)×25

The double-stranded PCR product was then transferred to E. coli DH5a using the in vivo homologous recombination method (Bubeck et al. 1993. Nucleic Acids Research 21, 3601-3602), establishing the Bsa1-free pUC19 plasmid.

### Ampicillin Screening

The ds-StoNuSeq harbours two unique Bsa1 restriction enzyme recognition sequences. Upon digestion the ds-StoNuSeq fragment was cloned into the Bsa1-free pUC19, and was transformed to E. coli DH5a. The resulting library was plated out on LA plates with varying concentrations of ampicillin (from 10 to 100 μg/mL).

mCherry Screening in E. coli DH5a

For screening by measuring mCherry fluorescence, three Bsa1 sites in the pHH100-mCherry plasmid had to be eliminated. By using the primer pairs V-Fwd-pHH100 (SEQ ID NO:31)/V-Rev-pHH100 (SEQ ID NO:32) and I-Fwd-pHH100 (SEQ ID NO:33)/I-Rev-pHH100 (SEQ ID NO:34) two PCR products were generated and were transferred to E. coli DH5a by using the in vivo homologous recombination method (Bubeck 1993 ibid). The resulting BsaI-free-pHH100 was amplified using the primer pair Bsa1-mCh-Fwd (SEQ ID NO:35)/Bsa1-mCh-Rev (SEQ ID NO:36). Upon Bsa1 digestion of both insert and vector, the insert was ligated into the vector and transferred to E. coli DH5a. The transformants carrying the constructed vector were plated out on LA-Kan (50 μg/mL) plates, creating the StoNuSeq-mCherry library.

mCherry Screening in Pseudomonas putida KT2440

For screening of expression in P. putida KT2440 cells, the ligation mixture used for mCherry screening in E. coli (described above) was also transferred to P. putida KT2440, by electroporation, similarly to the procedure described for E. coli DH5a (see above). Transformants carrying the constructed vector were selected on LA-Kan (50 μg/mL) plates, creating the StoNuSeq-mCherry library in P. putida.

### Primers Description:

SEQ ID NO: 28
Bsa1-Bla-Rev
SEQ ID NO: 29
Bsa1-Bla-Fwd

### Example 3

### Overview

Utilising the SUPERAPP technology we have created functional synthetic promoter-5′ UTRs, (ProU)s, in seven different microorganisms, across the bacterial and eukaryotic domains of life (Table 2).

### Gene Expression Measurement

For the determination of gene expression two main methods have been used: (i) agar-based assays, or (ii) fluorescent protein measurements.

The functional determination of gene expression from the reporter genes AMP, APR, CHL, KAN, KANT, TRP were performed on agar-based assays. Libraries of cells were plated on agar medium containing the antibiotics (AMP, APR, CHL, KAN, KANT) or amino-acid (TRP) based on the host-reporter gene. The growth of colonies on these medium indicate the presence of functional ProU sequences. In the case of S. albus and S. lividans cells were also plated on agar plates with higher concentrations of APR (250 and 500 μg/mL). Colonies identified on agar plates containing 250 and 500 μg/mL APR indicate stronger expression originating from the artificial ProU sequences.

Fluorescent-based measurements were performed with strains Escherichia coli and Pseudomonas putida expressing RFP. For these measurements colonies with visible red colours were picked from agar plates and were inoculated into 96-well microplates containing LB (with 50 μg/mL KAN) and were incubated overnight at 37° C. with 800 rpm agitation. After 18 hours of incubation the RFP expression levels were measured utilising a Tecan fluorescence microplate reader (Tecan).

TABLE 2
The list of hosts and reporter genes used for the identification of functional ProUs.

| | | Selection markers | | | | | | | |
|---|---|---|---|---|---|---|---|---|---|
| Hosts | | AMP | APR | CHL | KAN | KAN^{T} | RFP | YFP | TRP |
| B, G (−) | Escherichia coli | ✓ | ✓ | ✓ | ✓ | ✓ | ✓ | | |
| | Pseudomonas | | | | | | ✓ | | |
| | putida | | | | | | | | |
| | Thermus | | | | | ✓ | (✓) | | |
| | thermophilus | | | | | | | | |
| B, G (+) | Streptomyces | | ✓ | | | | (✓) | | |
| | albus | | | | | | | | |
| | Streptomyces | | ✓ | | | | (✓) | | |
| | lividans | | | | | | | | |
| | Corynebacterium | | | ✓ | | | (✓) | | |
| | glutamicum | | | | | | | | |
| E | Saccharomyces | | | | | | | (✓) | ✓ |
| | cerevisiae | | | | | | | | |

B, Bacteria; E, Eukaryote; G (−), Gram-negative; G (+), Gram-positive; AMP, ampicillin; APR, apramycin; CHL, chloramphenicol; KAN, kanamycin; KAN^{T}, thermostable kanamycin; RFP, red fluorescent protein; YFP, yellow fluorescent protein; TRP, tryptophan; ✓, confirmed; (✓), work in progress.

### Synthesis of Single-Stranded Random Nucleotide Sequences (RaNuSeq)

The synthetic DNA used in creating libraries was ordered from IDT (Belgium) as a four nmole Ultramer single-stranded DNA Oligo. Two different versions of the synthetic DNA was ordered: (A) this version has the fixed GGAG (SEQ ID NO:45) sequence in between the two stretches of random DNA, N(200) and N(7); (B) this version has only the random DNA that is of 200 nt, N(200) (FIG. 3).

The adapters on either end of random DNA provides two functions: Firstly, they are allowing the functional immobilisation of the oligo during the chemical synthesis; secondly, once the single stranded DNA is synthesised these adapters are used to generate the complementary strand by PCR. Adapters also harbour TypeIIS restriction enzyme recognition sequences (BsaI) that are utilised for various downstream applications. The placement of this random sequence directly upstream of the coding sequence has an important implication as it leads to the creation of both promoters and 5′ UTR sequences that are functional in combination with the coding sequence. In terms of adapter functionality, adapter 2 can be excised from the double stranded DNA and a gene specific adapter can be ligated depending on the coding sequence of the gene of interest. This allows the utilisation of synthetic DNA libraries for various constructs.

Generation of Double-Stranded RaNuSeq (ds-RaNuSeq)

The two adapter sequences on either end of DNA include the BioBrick prefix and suffix sequences, respectively (FIG. 3). The ds-RaNuSeq was generated by using the primers BB-Prefix-Fwd and BB-Suffix-Rev in 10 cycles of PCR. The low number of cycles was chosen to prevent the reduction of complexity of the initial ss-RaNuSeq during PCR amplification.

PCR parameters: (98−30)+[(98−10),(60−20),(72−20)]×10

TABLE 3
The synthetic DNA and the cloning method utilised
for the construction of DNA libraries.

| Hosts | Synthetic DNA design | Cloning method |
|---|---|---|
| Escherichia coli | A for RFP/B for AMP, | Gibson for RFP/ |
| | APR, CHL, KAN, | Bsal for the rest |
| | KAN^{T} | |
| Pseudomonas putida | A for RFP | Gibson |
| Thermus thermophilus | A for KAN^{T} | Gibson |
| Streptomyces albus | B for APR | Bsal |
| Streptomyces lividans | B for APR | Bsal |
| Corynebacterium | B for CHL | Bsal |
| glutamicum | | |
| Saccharomyces cerevisiae | B for TRP | Bsal |

AMP, ampicillin; APR, apramycin; CHL, chloramphenicol; KAN, kanamycin; KAN^{T}, thermostable kanamycin; RFP, red fluorescent protein; TRP, tryptophan.

Escherichia coli

In the construction of all libraries E. coli was used a cloning host. For the specific selection of dual-host functionality it was also used as an expression host except for the yeast specific TRP (Table 2).

E. coli cells were grown in Lysogeny broth (per L: 10 g tryptone, 5 g yeast extract, 10 g NaCl, 15 g agar for solid plates) at 37° C.

### DNA Manipulations—Plasmid/Vector Constructions

Two general approaches were used to create the libraries: BsaI- or Gibson assembly-based.

### BsaI Restriction Based Cloning

For this restriction and ligation based cloning strategy, the plasmid was amplified with a primer pair that excludes the promoter upstream of the gene of interest. At the same time, the primers introduce a type II restriction site which is recognized by BsaI. The restriction site overhangs are designed in a way that the restriction site itself will be cleaved off, leaving an NATG overhang at the start of the gene of interest and an overhang. Vector and insert was ligated using either T4 ligase or quick ligase and a vector to insert ration of 1:7.

### Gibson Assembly

Gibson assembly mix was made according to Gibson et al., 2009(DOI: 10.1038/nmeth.1318)

Use equimolar amounts (or 1:2 ratio) of both purified vector and insert was used (100 ng) and a volume of 5 μL, added to a volume of 15 μL of Gibson assembly mix.

In this procedure, the primer pairs exclude the natural promoter in front upstream of the gene of interest. The reverse primer also introduces the BB Prefix, upstream of the promoter region to be used as an homologue sequence Gibson assembly cloning.

For the downstream part, the library has to be fitted with an adapter that is specifically designed for the gene of interest.

### Adapter Attachment Protocol

Upon BsaI digestion the DNA library carries a NATG overhang (SEQ ID NO:75). Complementary oligos can be designed with suitable overhang which can be ligated. This adapter attachment allows Gibson assembly. Overhangs can be between 15 and 30 bp long.

### Phosphorylation of the Adapters

7 μL oligo 1 (700 pmol) use 100 μM primer dilution (100 μM (=100 pmol/μL))
7 μL oligo 2 (700 pmol) use 100 μM primer dilution (100 μM (=100 pmol/μL))

### 1.5 μL T4 Ligase Buffer

### 0.8 μL PNK

### Activation for 30 min at 37° C.

30 min at 65° C. for heat inactivation
Add 1 μL of 4 M NaCl and run the annealing program “Anneal” on the PCR2 machine.
Make dilutions up to 1:1000. Total amount is 1400 pmol/17.3 μL=80.9 pmol/μL undiluted
95° C. 10 m, 80° C. 2 m, 75° C. 2 m, 70° C. 3 m, 65° C. 5 m, 60° C. 10 m, 55° C. 10 m. 50° C. 5 m, 45° C. 3 m, 40° C. 2 m, 35° C. 1 m 30° C. 1 m, 25° C. 1 m, 20° C. 1 m, 4° C. hold.

### Ligation of the Gene Specific Adapter

### 5 μL Quick Ligase Buffer

˜30 ng (200 fmol) “vector” library del BsaI
1 μL of 1:100 dilution (800 fmol) “insert” adapter (1:100 dilution, gives a 1:4 molar ration vector to insert, results in 25 mM NaCl—commercial ligases can handle up to ˜70 mM NaCl)

### Quick Ligase 0.5 μL

### H₂O Fill to 10 μL

Incubate at RT for 5 min and then use desired amount for PCR (have used up to 5 μL)
PCR may be done with either Q5 or Taq, Taq adds some As, but these are removed in the assembly process:

### 10 μL Q5 Buffer

1 μL dNTPs
2.5 μL library_biobrick_del_F (SEQ ID NO:71)/AII_biobrick_F(SEQ ID NO:70)
2.5 μL rv adapter

### 0.5 μL Q5 PoI

### 5 μL Template (QL)

### 28.5 μL H₂O

### Run PCR (Iti):

### 98° C. 30 s

98° C. 10 s (do 10 cycles)
20° C. s (adjust this temperature to your primer pair)
72° C. 20 s (cycle ends)

### 72° C. 1 min

4° C. hold

Run on gel to verify the size (˜250 nt with 200N del BsaI library, depending on adapter size)

### Heat Shock Transformation

Take chemically competent DH5α E. coli, 100 μL aliquots from the −80° C. freezer (˜10⁶ cfu). Let chill on ice for 10 minutes. Add an amount 1-200 ng for a plasmid or about 40-50 ng DNA from a Gibson mix (half of the mix which in total should not be more than 100 ng).

### Incubate on ice for 30 min

Heat shock at 42° C. for 45 seconds
Chill on ice for 2-5 minutes
Add 900 μL LB media
Shake at 27° C. for one hour. Take 200 μL per medium agar plate or whole transformation batch (1 mL) for a big agar plate and spread out.
Incubate over night at 37° C.
Pseudomonas putida KT2440

P. putida cells were grown in Lysogeny Broth (or agar 15 g/L) at 30° C.

### Plasmid/Vector Detail

Plasmid DNA: pHH100 vector, which is suitable for gene expression in organisms E. coli and P. putida. Kanamycin resistance which is expressed in both host organisms and is used as a selective pressure. The plasmid also contains a gene that codes for an mCherry protein that can be expressed by both organisms.

### DNA Manipulations—Plasmid/Vector Construction

For this part of the work, Gibson assembly was used (see E. coli section for general procedure). The PCR uses the primer pair: Pp_pHH100_mCherry_F (SEQ ID NO:67) and Pp_pHH100_mCherry_R (SEQ ID NO:68) which creates a backbone that excludes the natural mCherry promoter and introduces the BioBrick Prefix sequence. Note that for a successful PCR, the linearized plasmid had to be used. The plasmid was digested using the NdeI site which is just upstream of the mCherry gene. The backbone was DpnI treated and purified and used in the Gibson assembly together with the suitable library. The Gibson mix is transferred into the cloning host E. coli via heat shock transformation and grown on LB plates containing kanamycin. The library size is about 3000 colonies per 10 μL Gibson mix. From this library, about 200 clones were picked that were appearing red to naked eye. The plasmids were isolated from the whole library and transferred into P. putida by electroporation.

### DNA Transformation Details

An overnight culture of the recipient strain was diluted in 1:100 in a rich medium with appropriate antibiotics. One mL of culture was used for every transformation. Cultures were grow at 30° C. with shaking for 2-4 h, until an OD600 ˜0.3. For each transformation, 1 mL of culture was pelleted in a microcentrifuge tube. Pellets were resuspended in 1 mL cold sterile water or 10% glycerol, and centrifuged once more and resuspended in 1 mL of water or 10% glycerol. Centrifuged once more and pellet was resuspended in ˜50 μL of cold sterile water or 10% glycerol.

### To Transform Electrocompetent Cells

Mix an aliquot (˜50-70 μL) of cells with 1-2 μL DNA in a low ionic strength buffer/water (dialyze or EtOH-precipitate ligations etc.)
Place in pre-chilled cuvette
Electroporate (2.5 kV for 2 mm gap or 1.8 V for 1 mm gap; 200Ω, 25 μF), Time constant should be ≥4 msec
Immediately add 1 mL of SOC or LB to cell/DNA mix and place in test tube
Grow at 30° C. for 1.5 hours
Plate onto selective media
Grow overnight at 30° C.

### Sequencing

For sequencing, plasmids were isolated with the Plasmid Miniprep kit from Qiagen and were sequenced using the primer Pp_pHH100_mCherry_Seq.

Thermus thermophilus

A knockout strain of Thermus thermophilus HB27 with high transformation efficiency (Genome accession number: AE017221), HB27Δago, was provided by Dr. José Berenguer (Universidad Autónoma de Madrid) and used in this study.

### Growth Conditions

Thermus Broth (TB) was used for T. thermophilus strain propagation and assays, which contains the following: bactotryptone (8 g/L), yeast extract (4 g/L) and NaCl (3 g/L), all dissolved in mineral water. 1.5% agar was added to the TB medium for growth on plates. A gradient range of Kanamycin (30 μg/ml, 60 μg/ml, 90 μg/ml) were added to the growth medium when selection is required.

Stock pellet of T. thermophilus from −20° C. was thawed at room temperature, before the addition of 1 mL of TB and in subsequent transferred to a 100-150 mL flask with 20 mL of TB. The strain was cultivated in a 65° C. shaker under mild shaking (150 rpm). To avoid plates from drying, TB plates were incubated in a moisturized Tupperware box in a 65° C. incubator.

### Plasmid/Vector Details

The E. coli/T thermophilus shuttle vector, μMK184 (José Berenguer, 2007, DOI: 10.1111/j.1365-2958.2007.05687.x), provides the thermostable Kanamycin resistance as the screening reporter.

### DNA Manipulations and Plasmid/Vector Construction

The pSLPa promoter of the thermostable kanamycin marker was excluded via σ PCR using the primers pair “Tt_pMK184_kan_F” (SEQ ID NO:61) and “Tt_pMK184_kan_R” (SEQ ID NO:62). The PCR was carried out with CloneAMP™ HiFi PCR premix (Takara Bio, Inc.) and reagents concentrations, Tm were set (55° C.) according to the manufacturer instructions.

The site of the original promoter is replaced with a randomized sequence library via Gibson assembly. A BioBrick Prefix homology site is introduced via the overhang of the reverse primer on the vector, which assemble to one end of the randomized sequence library. On the other end, the adaptor-attached randomized sequence library contains around 20 nucleotide homologies to the reporter gene. Gibson assembly of the promoter-less vector and adaptor-attached randomized sequence library was carried out using a home-made Gibson reaction mix (Gibson et al., 2009, Nature, DOI: 10.1038/nmeth.1318). The vector/Insert ratio was adjusted by sequence length to 1:1 and up to a 100 ng of DNA was transformed to E. coli strain DH5α, which serves as the cloning host and used for the first round of synthetic promoters screening. A Shine-Dalgarno sequences containing library was used in this screen.

### DNA Transformation Details

Overnight T. thermophilus strain culture was re-inoculated in fresh pre-warmed TB medium at a dilution of one over fifty. The strain was cultivated in a 65° C. shaker until an OD_{550 nm} of 0.4 was reached. 0.8 mL of the culture was aliquoted to a new 12 mL tube, follow by an addition of 200 ng of plasmids. The mixture was incubated further at 65° C. with shaking for 4 hours, after which it was plated on selective TB plates. Colonies were isolated after an overnight incubation at 65° C.

### Phenotypic Screening and Promoter Activity Confirmations

To isolate cross species synthetic promoters between E. coli and T. thermophilus, 10 out of 20 μL of the Gibson assembly mix was first chemically transformed to E. coli DH5α under a Kanamycin selection of 50 μg/mL. Around 1,500 colonies were obtained with 3 to 4 rounds of transformations. The cell lawn was then scraped and the plasmids were isolated with QIAprep Spin Miniprep Kit (Qiagen) according to the manufacturers and later quantified with Nanodrop.

200 ng of the plasmids mix with functional promoters from E. coli were transformed to T. thermophilus. The transformants were challenged by a series of kanamycin concentrations (30 μg/ml, 60 μg/ml, 90 μg/ml) and the resulted colonies were picked and further purified with streaking.

### Sequencing

Purified colonies were re-streaked on TB agar with the basal kanamycin selection (30 μg/mL). After an overnight incubation, the cell lawn was scraped and the plasmid was isolated with QIAprep Spin Miniprep Kit (Qiagen). The plasmid and a reverse sequencing primer, “Tt_pMK184_seq” (SEQ ID NO:63), was together sent for LIGHTRUN sequencing (GATC Biotech). The obtained sequences were checked and analysed using Genome Compiler software.

### Streptomyces

### Bacterial Strains and Growth Conditions

The bacterial strains used in this study are listed in Table 4.

TABLE 4
Bacterial strains and plasmids used in this study.

| Bacterial strains and | | |
|---|---|---|
| plasmids | Description | Reference |
| S. albus J1074 | Derivative of S. albus DSMZ | Chater and Wilde, 1980 |
| | 40313, isoleucine and valine | DOI: 10.1099/00221287- |
| | auxotrophic, deficient of | 116-2-323 |
| | Sa/GI-based restriction | |
| | modification system. Widely | |
| | used for heterologous | |
| | protein/antibiotic production. | |
| S. lividans TK24 | Plasmid-free derivative | Hopwood et al., 1982 |
| | strain of S. lividans 66. | doi: 10.1099/00221287-129- |
| | Model Streptomyces strain | 7-2257 |
| | routinely used for heterologous protein | |
| | production. | |
| E. coli DH5α | Standard cloning. | |
| E. coli S17.1 | Conjugative transfer of DNA | |
| | to Streptomyces spp. | |
| E. coli C2984 | High efficiency turbo | NEB |
| | competent cells for library | |
| | construction. | |
| pKC1218 | Conjugative E. coli-Streptomyces | Bierman et al., 1982 |
| | shuttle vector. | https://doi.org/10.1016/0378- |
| | SCP2* Streptomyces | 1119(92)90627-2 |
| | replicon, pMB1 E. coli | |
| | replicon, aac(3)IV gene | |
| | conferring apramycin | |
| | resistance. | |
| pKC1218_kan | pKC1218-derivative with | This study. |
| | inserted aph(3′) cassette | |
| | between oriT and SCP2* | |
| | conferring kanamycin | |
| | resistance. | |
| pKC1218_kan-200N | pKC1218_kan with 200N | This study. |
| | library inserted directly | |
| | upstream of aac(3)IV start | |
| | codon. | |
| pKC-P | pKC1218_kan with | This study. |
| | promoterless aac(3)IV gene. | |

E. coli strains were grown in Lysogeny broth. When required, antibiotics were added to cultures at the following concentrations: 50 μg/mL kanamycin, 50 μg/mL apramycin. For sporulation S. lividans TK24 was grown at 30° C. on ISP4 agar (BD Difco ISP medium 4), S. albus J1074 at 30° C. on soy flour mannitol agar (20 g/L soy flour, 20 g/L mannitol, 20 g/L agar). Conjugation reactions were performed using the same media supplemented with 10 mM MgCl₂ at 30° C.

### Recombinant DNA Techniques

Plasmid DNA from E. coli was isolated using standard protocols. Restriction enzymes and molecular biology reagents were used according to the manufacturers' instructions (NEB, England).

TABLE 5
Primers used in this study.

| | Sequence | |
|---|---|---|
| Primers | 5′-3′ | Purpose |
| pKC_fwd_kan | SEQ ID | Cloning the aph(3′) gene into pKC1218. |
| | NO: 37 | |
| pKC_rev_kan | SEQ ID | Cloning the aph(3′) gene into pKC1218. |
| | NO: 38 | |
| Bsal_Apr5f_new | SEQ ID | Introduction of Bsal restriction sites for |
| | NO: 39 | cloning of 200N library upstream of |
| | | aac(3)IV start codon. |
| Bsal_oriV3r | SEQ ID | Introduction of Bsal restriction sites for |
| | NO: 40 | cloning of 200N library upstream of |
| | | aac(3)IV start codon. |
| BB_Prefix_Fwd | SEQ ID | Amplification of 200N library (without |
| | NO: 24 | SD) for cloning upstream of promoter-less |
| | | aac(3)IV gene. |
| BB_Suffix_Rev | SEQ ID | Amplification of 200N library (without |
| | NO: 25 | SD) for cloning upstream of promoter-less |
| | | aac(3)IV gene. |
| 5511-F | SEQ ID | Colony PCR |
| | NO: 41 | |
| 234_R | SEQ ID | Colony PCR |
| | NO: 42 | |
| 5654_F | SEQ ID | Sequencing primer |
| | NO: 43 | |
| apr_5c | SEQ ID | Sequencing primer |
| | NO: 44 | |

Construction of DNA in E. coli

For selection of functional synthetic promoters/UTRs in E. coli and Streptomyces, a shuttle expression plasmid based on pKC1218 (Table 1), a conjugative vector with low copy Streptomyces replicon (SCP2* rep), pMB1 E. coli replicon and apramycin resistance cassette (aac(3)IV) was modified as follows.

The pKC1218 backbone was amplified using primers pKC_rev_kan and pKC_fwd_kan (Table 5) introducing 40 bp homology overhangs to a 1100 bp SalI-fragment of pTA16 containing the aph(3′) kanamycin resistance gene, and the cassette was cloned between oriT and SCP2 replicon by in vivo homologous recombination in E. coli DH5α (Bubeck et al. 1993, Nucleic Acids Research, 21, 3601-3602, yielding pKC1218_kan.

pKC1218_kan was linearized by PCR using primers BsaI_Apr5f_new and BsaI_oriV3r (Table 5) introducing BsaI-overhangs on both ends. The product was phosphorylated using T4 PNK and re-ligated and the circularized product digested with BsaI. A library of 200 nucleotide long random sequences was amplified by PCR using primers BB_Prefix_Fwd and BB_Suffix_Rev (Table 2), digested with BsaI and ligated directly upstream of the promoterless aac(3)IV gene, creating a library of 200 nucleotide long randomized promoter plus 5′ UTR sequences in pKC1218_kan. This library was then transferred to turbo competent E. coli C2984 cells (NEB) by chemical transformation. Transformants were selected on 14 cm agar plates containing LA supplemented with 50 μg/mL kanamycin. The entire population of obtained transformants (lawn on plate) was pooled and plasmid DNA isolated using QuiaPrep Spin Miniprep Kit (Qiagen) according to the manufacturers' instructions. 1 μg of plasmid DNA was used to transform chemically competent E. coli S17.1 cells for conjugal transfer of the library to Streptomyces. S17.1 transformants were selected on LA medium supplemented with 50 μg/mL kanamycin. To select for promoters/UTRs functional both in E. coli and Streptomyces, transformants were also selected on LA supplemented with 50 μg/m apramycin (Lib Am).

Selection of Functional Promoters/UTRs in S. albus J1074 and S. lividans TK24

For selection of functional synthetic promoters/UTRs based on antibiotic resistance (apramycin) phenotype, the 200N-library was transferred from E. coli S17.1 to S. albus J1074 and S. lividans TK24 by intergenic conjugation. Conjugation reactions were performed as described previously (Kieser et al., 2000) with minor modifications. S17.1 library transformants (ca. 20.000 transformants for LibAm and Lib Km, respectively) were pooled by adding 3 mL LB medium to a 14 cm agar plate and the cells brought into suspension using a sterile glass rod. 100 μL of the obtained suspensions was used to inoculate 25 mL LB medium and incubated at 37° C. for ca. 2.5 h until the cells had reached an OD₆₀₀ of 0.4. The cells were centrifuged at 2.000×g for 5 min at room temperature, and the obtained pellet re-suspend in 2 mL fresh LB medium and placed on ice. Spore suspensions of two freshly sporulated plates of S. albus and S. lividans were prepared using 4 mL sdH₂O and the obtained suspensions filtered through sterile cotton wool. 50 μl of these spore suspensions were added to 500 μl 2×YT medium (16 g/L Tryptone, 10 g/L, Yeast Extract, 5 g/L NaCl) and incubated at 50° C. for 5 min to induce germination. The spore suspensions were cooled under running water before 500 μl of E. coli suspension was added, and the resulting suspension was mixed by inversion before spreading it onto two 9 cm agar plates (SFM+10 mM MgCl₂ for S. albus, and ISP4+10 mM MgCl₂ for S. lividans). Conjugation plates were incubated at 30° C. for 14-16 h before overlaying them with antibiotic solutions yielding final concentrations of 30 μg/mL nalidixic acid and 50/250/500 μg/mL apramycin in agar media. The plates were then further incubated at 30° C. until exconjugants appeared (2-3 d). Single exconjugant colonies were transferred to fresh plates supplemented with corresponding concentrations of apramycin for further analysis by colony PCR and sequencing of the 200N region.

### Sequence Analysis of Functional Synthetic Apramycin Promoters/UTRs

Single colonies of exconjugants were subjected to colony PCR using primers 5511_F and 234_R (Table 2) amplifying a 710 bp fragment surrounding the 200N region. Single colonies were picked into 100 mL of 200 mM lithium acetate and 1% SDS and incubated at 70° C. for 5 min. 300 mL of 96% ethanol were added, the suspension vortexed and centrifuged at 15.000×g for 3 minutes. The pellet was washed with 70% ethanol, dried and dissolved in 20 mL sterile deionized water. 1 mL of the resulting solution was used as template for PCR reactions using Taq polymerase (NEB). Amplicons of the expected size were extracted from 0.8% agarose gels and purified using the QiaQuick gel extraction kit (Qiagen) according to the manufacturers' instructions, and send for sequencing using sequencing primers 5654_F or apr_5c (Table 2).
Results
   Screening of 200N Library for Functional Promoters/UTRs in S. albus J1074

TABLE 6
Number of exconjugants obtained per plate after transferring
the 200N library (pKC1218_kan-200N) to S. albus J1074
and screening for functional promoters/UTRs using different
concentrations of apramycin (cfu/plate).

| | | Overlay | Overlay | Overlay | Overlay |
|---|---|---|---|---|---|
| Library | Dilution | with Km | with Am | with Am | with Am |
| type | plated | 50 | 50 | 500 | 2000 |
| Lib Km | 0 | 420 | 220 | 2 | 0 |
| Lib Km | 10⁻¹ | 31 | 91 | 1 | 0 |
| Lib Km | 10⁻² | 1 | 3 | 0 | 0 |
| Lib Km | 10⁻³ | 0 | 0 | 0 | 0 |
| Lib Am | 0 | 800 | 640 | 6 | 0 |
| Lib Am | 10⁻¹ | 30 | 180 | 7 | 0 |
| Lib Am | 10⁻² | 2 | 8 | 0 | 0 |
| Lib Am | 10⁻³ | 0 | 0 | 0 | 0 |

Lib Km = transfer of 200N library from C2984 to S17.1, selection of transformants with Km50; Lib Am = transfer of 200N library from C2984 to S17.1, selection of transformants with Am50.

200N exconjugants for S. albus J1074 were successfully obtained upon selection with up to 500 μg/mL apramycin, while cells carrying pKC1218_kan with the wild-type apramycin promoter were only able to grow on medium supplemented with up to 50 μg/mL apramycin. The apramycin promoterless version of pKC1218_kan served as a negative control and S. albus exconjugants carrying this plasmid were not obtained when selecting with 50 μg/mL apramycin.

Screening of 200N Library for Functional Promoters/UTRs in S. lividans TK24

200N exconjugants for S. lividans were successfully obtained upon selection with up to 500 μg/mL apramycin. Exconjugants carrying pKC1218_kan with the wild-type apramycin promoter were also able to grow on medium supplemented with up to 500 μg/mL apramycin. The apramycin promoterless version of pKC1218_kan supported growth of S. lividans exconjugants when selected with up to 50 μg/mL but not at any higher concentration tested (250 μg/mL and 500 μg/ml).

TABLE 7
Number of exconjugants obtained per plate after transferring
the 200N library (pKC1218_kan-200N) to S. lividans
TK24 and screening for functional promoters/UTRs using
different concentrations of apramycin (cfu/plate).

| | | Overlay | Overlay | Overlay | Overlay |
|---|---|---|---|---|---|
| Library | Dilution | with Km | with Am | with Am | with Am |
| type | plated | 50 | 50 | 500 | 2000 |
| Lib Km | 0 | | 19 | 80 | 30 |
| Lib Km | 10⁻¹ | | 135 | 61 | 23 |
| Lib Am | 0 | | 0 | 50 | 255 |
| Lib Am | 10⁻¹ | | 325 | 200 | 122 |

Lib Km = transfer of 200N library from C2984 to S17.1, selection of transformants with Km50; Lib Am = transfer of 200N library from C2984 to S17.1, selection of transformants with Am50.

TABLE 8
Transfer of pKC1218_kan (with native apramycin promoter)
and pKC-P (=PCR-amplified, phosphorylated and religated
pKC1218_kan without AM promoter) from S17.1 to S. albus
and S. lividans, (cfu/plate).

| | Km50 | Am50 | Am250 | Am500 |
|---|---|---|---|---|
| S. albus/pKC1218_kan, | 6400 | 7000 | 0 | 0 |
| 10⁰ | | | | |
| S. albus/pKC1218_kan, | 105 | 300 | 0 | 0 |
| 10⁻¹ | | | | |
| S. albus/pKC-P, | >>6400 | 0 | 0 | 0 |
| 10⁰ | | | | |
| S. albus/pKC-P, | 300 | 0 | 0 | 0 |
| 10⁻¹ | | | | |
| S. lividans/pKC1218_kan, | 240 | 200 | 160 | 100 |
| 10⁰ | | | | |
| S. lividans/pKC1218_kan, | 7 | 3 | 2 | 0 |
| 10⁻¹ | | | | |
| S. lividans/pKC-P, 10⁰ | 2500 | 2500 | 0 | 0 |
| S. lividans/pKC-P, 10⁻¹ | 450 | 25 | 0 | 0 |

Corynebacterium glutamicum

C. glutamicum wild type ATCC 13032 cells were grown in Brain heart infusion medium (Brain heart infusion mix: 37 g/L, 91 g/L of sorbitol [in broth medium only] at 30° C.

### DNA Manipulations—Plasmid/Vector Construction

BsaI Restriction Based Cloning in Vector pXMJ19.

pXMJ19 is a shuttle vector that is suitable for replication in E. coli and C. glutamicum. The chloramphenicol resistance gene works in both organisms. Amplification of the whole plasmid under exclusion of CHL promoter with the primer pair: Cg_pXMJ19_Chl_F and Cg_pXMJ19_Chl_R. This same primer pair introduces BsaI restriction sites as overhangs and creates a backbone of ˜4300 nt. After the PCR, the product is digested with DpnI and BsaI in CutSmart at 37 degree for 3 hrs and cleaned up. The library is being amplified (without SD) and cut with BsaI and purified. For the T4 ligation over night at 16 degree use: 60 ng of backbone and 20 ng of insert (1:7 backbone to insert ratio). Heat inactivate the next day and use 10 μL for transformation into E. coli gives ˜1000 transformants/10 μL. library size total ˜2000.

### DNA Transformation Details

For cloning in E. coli was used following the stranded heat shock protocol for chemical competent cells. For DNA transformation to C. glutamicum, follow the protocol was used:

Inoculate 5 mL BHIS (Brain Heart infusion) and incubate at 30° C. O/N
Inoculate 25 mL BHIS with 500 μl pre-culture
Incubate at 30° C. until OD600=˜1.5
Centrifuge at 4500 rpm for 3-5 min at 4° C. Discard supernatant
Wash cells 2× in 25 mL cold TG buffer (10% glycerol, 1 mM Tris)
Centrifuge at 4500 rpm for 3-5 min at 4° C. Discard supernatant
Wash cells 2× in 25 mL cold 10% glycerol
Centrifuge at 4500 rpm for 3-5 min at 4° C. Discard supernatant
Re-suspend cell in back-flow (˜400 μl) and keep on ice
Add 100 μl of cells and up to 1 μg DNA to cold electroporation cuvette

### Electroporate at 2.5 kV, 25 μF

Add cells to 4 mL BHIS (preheated at 46° C.)
Incubate cells at 46° C. for 6 min
Incubate cells at 37° C. for 1 hr
Incubate cells at 30° C. for 30 min
Centrifuge at 4500 rpm for 3-5 min and plate on selection media
Incubate at 30° C. for 2 days

### Phenotypic Screening and Promoter Activity Confirmations

Screening for expression of CHL resistance gene on CHL 15 plates. Library size in E. coli was ˜2000, going down to ˜200 in Cg.

### Sequencing

Plasmids were isolated using the plasmid miniprep kit from QIAGEN. Since C. glutamicum is a gram positive organism, an extra step is required: Make a solution of P1 buffer+15 mg/mL final concentration of lysozyme. Use 350 μL of that to incubate with the colony for 3 hours at 37 degree. Plasmid concentrations usually too low to send for sequencing->retransformation into E. coli via heat shock, then isolation from there. For sequencing use the primer: Cg_pXMJ19_Chl_Seq (SEQ ID NO:64).

Saccharomyces cerevisiae

### Growth Conditions;

### Growth Medium:

YPD: yeast extract peptone dextrose ready-to-use from Sigma-Aldrich. Use: 50 g in 1 L of distilled water. Autoclave for 15 minutes at 121° C. For agar plates, add 15 g/l.
Contains (g/L):
Bacteriological peptone, 20
Yeast extract, 10

### Glucose, 20

For yeast cultivation prior to transfection use 2×YPD

### Drop Out Media:

Make a 10× concentrated stock solution by stirring to suspend 6.8 g yeast nitrogen base powder (Sigma), 5 g glucose, and 5-10 mg of appropriate amino acids in 100 mL water. Warm if necessary to aid solubilization. Filter sterilize and store at 2-8° C. Appropriate amino acids: Add 1.92 g/L of Yeast Synthetic Drop-Out Media Supplements without tryptophan (Sigma)

Dilute the 10× concentrated stock to a 1× working solution by adding 100 mL concentrated stock to 900 mL sterile water. For plates: Autoclave water plus 15 g/L agar together, add from 10× sterile stock appropriate amount afterwards.

Growth temperature: 30° C.

### DNA Manipulations—Plasmid/Vector Construction(s);

Cloning in E. coli. The vector used was pENZ004 provided by Sara Castaño Cerezo from the Institut National des Sciences Appliquées de Toulouse|INSA Toulouse, Biosystems and Process Engineering Laboratory (LISBP). pENZ004 is a shuttle vector suitable for cloning in E. coli, which also contains homologous regions for integration into the Site 2× chromosome locus in S. cerevesia. For Gibson cloning, the vector was amplified using primer pair Sc_pENZ004_G_trp_F (SEQ ID NO:52) and Sc_pENZ004_G_trp_R (SEQ ID NO:53). The adapter for the library was created using primer pair Adapter_pENZ004_trp_UpS (SEQ ID NO:73) and Adapter_pENZ004_trp_LoS (SEQ ID NO:74). The adapter library then was amplified using AII_biobrick_F (SEQ ID NO:70) and Adapter_pENZ004_trp_LoS For BsaI cloning, the vector was amplified using primer pair Sc_pENZ004_B_trp_F (SEQ ID NO:57) and Sc_pENZ004_B_trp_R (SEQ ID NO:58).

There is no direct screening of the library in E. coli, as they are being selected for the plasmid on the plasmid ampicillin resistance.

### DNA Transformation Details;

Homologous recombination in yeast (inspired by Belden et al, 2015, Journal of Microbiological Methods https://doi.org/10.1016/j.mimet.2013.11.013 and Gietz, ₂₀₁₄, Methods in Molecular Biology https://doi.org/10.1007/978-1-4939-0799-1_4)
Inoculate the yeast strain into 5 mL of liquid medium (2×YPD) and incubate overnight on a rotary shaker at 200 rpm and 30° C.
Cut desired plasmid to linearize. Use 100 ng and use dirty digestion mix for transformation. Here, cut with Sfil in CutSmart at 50 degree.
Determine the titer of the yeast culture: Pipet 100 μL of cells into 900 μL of 2×YPD in a spectrophotometer cuvet, mix thoroughly by inversion, and measure the OD at 600 nm. An OD600 of 1 equals 3×10⁷ cells/mL.
Add cells to 25 mL of pre-warmed 2×YPD to a titer of 5×10⁶ cells/mL, grow for about 4 hrs at 30 degree, shaking until titer reaches 2×10⁷ cells/mL.
Harvest the cells by centrifugation at 3000 g for 5 min, wash the pellet twice in 25 mL of sterile water, and resuspend the cells in 1.0 mL of 0.1 M LiOAc.
Transfer the cell suspension to a 1.5-mL microcentrifuge tube, centrifuge for 30 s, and discard the supernatant.
Resuspend the cells in 100 μL per 1×10⁸ cells total of 0.1 M LiOAc and pipet samples of 50 μL (about 5×10⁷ cells into aliquots. Keep aliquots on bench top until use or in fridge for up to one week.
Prepare the T Mix (can be done beforehand and kept on ice/water until usage):
Dissolve 2 mg of salmon sperm DNA (Sigma) in 1 mL of TE: 10 mM Tris-HCl, 1 mM Na2 EDTA pH 8.0, using a stir plate overnight at 4° C.
Denature an appropriate sample size of carrier DNA in a boiling water bath for 5 min and chill immediately in an ice/water bath.
Add together:
240 μL PEG 3500 (50% [w/v])

### 36 μL LiAc 1.0 M

50 μL SS carrier DNA (2.0 mg/mL), denaturated
34 μL Digested plasmid DNA (100 ng)
360 μL Total volume (excluding cells)
Note: Vortex the carrier DNA before pipetting it
Centrifuge yeast cells at top speed for 30 s, remove supernatant.
Add 360 μL of T Mix to each transformation tube and resuspend the cells by vortex mixing vigorously.
Place the tubes in a shaker at 30 degree for 30 min.
Place the tubes in a floating rack and incubate them in a water bath at 42° C. for 30 min. Invert every 5 minutes.
Microcentrifuge the tubes at top speed for 30 s and remove the T Mix carefully and completely with a micropipettor.
Pipet 400 μL of sterile water into the transformation tube. Dissolve the pellet with a sterile micropipette tip to resuspend the cells.
Incubate the plates at 30° C. for 3-4 d and count the number of transformants.

### Phenotypic Screening and Promoter Activity Confirmations;

Phenotype screening for growth on drop out media. Library size in cloning host was in 10⁴ range, in yeast then to 10\hat{ }3.
Details on sequencing;
Crude yeast genomic DNA extraction was performed with the following procedure (Kristjuhan et al (2011, Biotechniques, doi: 10.2144/000113672)):

### Reagents

1. 0.2 M Lithium acetate 1% SDS solution.

### 2. Ethanol 96-100% and 70%.

### Procedure

1. Pick one yeast colony from the plate or spin down 100-200 μl of liquid yeast culture (OD600=0.4). Suspend cells in 100 μl of 200 mM LiOAc, 1% SDS solution.
2. Incubate for 5 minutes at 70° C.
3. Add 300 μl of 96-100% ethanol, vortex.
4. Spin down DNA and cell debris at 15 000 g for 3 minutes.
5. Wash pellet with 70% ethanol
6. Dissolve pellet in 100 μl of H2O or TE and spin down cell debris for 15 seconds at 15 000 g.
7. Use 1 μl of supernatant for PCR.

The PCR creates a 2000 nt piece from the upstream homologous region to the downstream homologous region using primer pair: Sc_pENZ004_Hom_F (SEQ ID NO:54) and Sc_pENZ004_Hom_R (SEQ ID NO:55). This piece was purified and then sent for sequence analysis with an internal primer: Sc_pENZ004_Seq (SEQ ID NO:56).

A number of promoters/5′ UTR sequences were identified for C. glutamicum (10 sequences), E. coli (146 sequences), P. putida KT2440 (11 sequences), S. cerevisiae (10 sequences), S. albus (16 sequences), S lividans (8 sequences) and T. thermophilus (11 sequences) as described above. A selection of the sequences identified for each organism is provided in Tables 9-15 below.

TABLE 9
C. glutamicum sequences

| Clone | SEQ ID NO |
|---|---|
| 22EF79 | 77 |
| 22EF75 | 78 |
| 22EF83 | 79 |
| 22EF80 | 80 |
| 22EF97 | 81 |

C. glutamicum sequences identified using a Chloramphenicol reporter gene.

TABLE 10
E. coli sequences

| Clone | Fluorescence measurement (au) | SEQ ID NO |
|---|---|---|
| E6 | 2246 | 82 |
| F3 | 8034 | 83 |
| H5 | 12237 | 84 |
| J4 | 30254 | 85 |
| O3 | 59961 | 86 |

E. coli sequences identified using an mCherry reporter gene. Fluorescence measurements for each clone are shown as an indication of the expression level seen for each clone

TABLE 11
P. putida KT 2440 sequences

| Clone | SEQ ID NO: |
|---|---|
| 22EF90 | 87 |
| 22EF86 | 88 |
| 22EF88 | 89 |
| 22EF87 | 90 |
| 22EF85 | 91 |

P. putida KT2440 sequences identified using an mCherry reporter gene.

TABLE 12
S. cerevisiae sequences

| Clone | SEQ ID NO |
|---|---|
| 22EF21 | 92 |
| 22EF23 | 93 |
| 22EF25 | 94 |
| 22EF27 | 95 |
| 22EF29 | 96 |

S. cerevisiae sequences identified using a TRP1 reporter gene.

TABLE 13
S. albus sequences

| Clone | Apramycin concentration used for selection | | SEQ ID NO: |
|---|---|---|---|
| 1 | 50 | μg/ml | 97 |
| 2 | 50 | μg/ml | 98 |
| 3 | 50 | μg/ml | 99 |
| 4 | 50 | μg/ml | 100 |
| 2 | 500 | μg/ml | 101 |

S. albus sequences identified using an aac(3)IV reporter gene. Concentrations of apramycin used in the selection of clones are shown.

TABLE 14
S. lividans sequences

| Clone | Apramycin concentration used for selection | SEQ ID NO: |
|---|---|---|
| 1 | 500 μg/ml | 102 |
| 2 | 500 μg/ml | 103 |
| 3 | 500 μg/ml | 104 |
| 4 | 500 μg/ml | 105 |
| 5 | 500 μg/ml | 106 |

S. lividans sequences identified using an aac(3)IV reporter gene. Concentrations of apramycin used in the selection of clones are shown.

TABLE 15
T. thermophilus sequences

| Clone | SEQ ID NO: |
|---|---|
| K60_C1 | 107 |
| K90_C4 | 108 |
| K60_C5 | 109 |
| K90_C5 | 110 |
| K60_C20 | 111 |

T. thermophilus sequences identified using a thermostable kanamycin resistance reporter gene.

1. A method of identifying a factor which modulates gene expression, said method comprising:
a. providing a library of at least 1×10⁶ nucleic acid molecules, wherein each nucleic acid molecule comprises a stochastic sequence of at least 50 nucleotides, optionally wherein each nucleic acid molecule also comprises an identical pre-determined and fixed sequence either adjacent to or within the stochastic sequence, wherein said pre-determined and fixed sequence does not include a promoter sequence itself capable of initiating transcription of a gene;
b. introducing nucleic acid molecules from said nucleic acid library of (a) into nucleic acid constructs to prepare a library of test constructs, wherein each test construct comprises a nucleic acid molecule of part (a) upstream of a reporter sequence, and wherein each test construct does not comprise a further separate sequence upstream of the reporter sequence that can modulate expression of the reporter sequence;
c. introducing test constructs from said test construct library of (b) into host cells to prepare a library of host cells, wherein host cells in the host cells library comprise a test construct;
d. assessing expression of the reporter sequence in host cells from the library of host cells; and
e. based on the level of expression of the reporter sequence, identifying a factor which modulates gene expression.

2. The method of claim 1, wherein to prepare the library of host cells in step (c), host cells are contacted with the test construct library of (b).

3. The method of claim 1 or claim 2, wherein in step (d) expression of the reporter sequence takes place under a test condition.

4. The method of any one of claims 1 to 3, wherein step (e) comprises identifying the nucleic acid molecule of the test construct from a host cell having a level of expression of the reporter sequence that is of interest and/or identifying a test condition under which a host cell expresses the reporter sequence at a level of interest, thereby to identify a factor which modulates gene expression.

5. The method of any one of claims 1 to 4, wherein the factor is a genetic element, a regulatory molecule present in the host cell, or a test condition.

6. The method of any one of claims 1 to 5, wherein the factor is a genetic element and the identifying of step (e) comprises sequencing the stochastic sequence.

7. The method of any one of claims 1 to 6, wherein the method is for identifying an element which modulates gene expression and wherein the element is a regulatory sequence which regulates gene expression, a binding site for a regulatory molecule, a binding site for an enzyme, a sequence which interacts with an expression-modulating factor, or a sequence responsive to a condition which modulates gene expression.

8. The method of claim 7, wherein the element is a transcriptional and/or translational control sequence, or a sequence which interacts with a transcription and/or translation modulating factor or a sequence which is transcribed into RNA and affects the stability and/or function of the RNA transcript.

9. The method of claim 7 or 8, wherein said element is a promoter, enhancer, silencer, operator region, a 5′ UTR sequence, a ribosome binding site, a polymerase binding site, a binding site for an inducer or repressor of transcription, or a binding site for a transcription factor or any other factor which directly or indirectly modulates gene expression.

10. The method of any one of claims 1 to 9 wherein said method is a method of identifying an element which modulates gene expression under a test condition, wherein step (d) comprises:
(i) incubating the library of host cells under a test condition; and
(ii) assessing expression of the reporter sequence in host cells from the library of host cells;
wherein the nucleic acid molecule identified in step (e) is an element which modulates gene expression under a test condition.

11. The method of any one of claims 1 to 4, wherein said method is a method of identifying a test condition which modulates gene expression, wherein step (d) comprises:
(i) incubating the library of host cells under a test condition; and
(ii) assessing expression of the reporter sequence in host cells from the library of host cells;
wherein step (e) comprises identifying a nucleic acid molecule of the test construct from a host cell which expresses the reporter sequence under the test condition, and thereby identifying whether the test condition modulates gene expression.

12. A method of identifying a test condition which modulates gene expression, said method comprising performing steps (a)-(d) as defined in claim 1 or 2, wherein step (d) comprises:
(i) incubating the library of host cells under a test condition; and
(ii) assessing expression of the reporter sequence in host cells from the library of host cells;
and wherein said method further comprises identifying whether the test condition modulates gene expression.

13. The method of any one of claims 1 to 12, wherein the stochastic sequence in each nucleic acid molecule comprises one or more biased random sequences.

14. The method of claim 13, wherein said one or more biased random sequences is A/T rich.

15. The method of claim 14, wherein each nucleic acid molecule comprises A/T rich sequences at the −35 and/or −10 regions.

16. The method of any one of claims 1 to 15, wherein the stochastic sequence in each nucleic acid molecule comprises an identical fixed ribosomal binding site or part thereof.

17. The method of claim 16, wherein said fixed sequence is a translation-modulating sequence, preferably a Shine-Dalgarno sequence or a Kozak sequence.

18. The method of any one of claims 10 to 17, wherein the test condition is selected from any one of temperature, pH, oxygen saturation and/or light, or the presence or absence of a molecule or added factor, e.g. a chemical factor.

19. The method of any one of claims 1 to 18, wherein the library of host cells is incubated in or on a growth medium supplemented with one or more chemicals.

20. The method of claim 19, wherein said chemical is a sugar, amino acid, peptide, protein, antibiotic, a putative regulatory compound, or a test candidate compound.

21. The method of any one of claims 6 to 20, wherein the element is for modulating gene expression in a prokaryote, preferably P. putida or E. coli.

22. The method of any one of claims 6 to 20, wherein the element is for modulating gene expression in a eukaryote, preferably yeast and algae.

23. The method of any one of claims 1 to 22, wherein the reporter sequence is or comprises a reporter gene which is an antimicrobial resistance gene, and wherein assessing gene expression comprises contacting the host cells with an antibiotic.

24. The method of any one of claims 1 to 22, wherein the reporter sequence is or comprises a reporter gene which encodes a protein that is or generates a chromogenic marker, preferably a fluorescent marker, and wherein assessing gene expression comprises detecting a host cell comprising said chromogenic marker.

25. The method of claim 24, wherein the reporter gene encodes a fluorescent protein, preferably YRP, RFP or GFP, mCherry or luciferase.

26. The method of claim 24 or 25 wherein detection comprises flow cytometry.

27. The method of any one of claims 1 to 26, wherein the library of nucleic acid molecules comprises at least 1×10⁹, 1×10¹², 1×10¹⁵ or 1×10¹⁸ nucleic acid molecules.

28. The method of any one of claims 1 to 27, wherein the library of host cells comprises at least 1×10⁴, 1×10⁶, 1×10⁸, 1×10¹⁰ or 1×10¹² host cells, or wherein gene expression is assessed in at least 1×10⁴, 1×10⁶, 1×10⁸, 1×10¹⁰ or 1×10¹² host cells.

29. A library of nucleic acid molecules for use in a method as defined in any one of claims 1 to 28 for identifying a factor which modulates gene expression, wherein said library comprises at least 1×10⁶ nucleic acid molecules, wherein each nucleic acid molecule comprises a stochastic sequence of at least 50 nucleotides, optionally wherein each nucleic acid molecule also comprises an identical pre-determined and fixed sequence either adjacent or within the stochastic sequence, wherein said pre-determined and fixed sequence does not include a promoter sequence itself capable of initiating transcription of a gene.

30. The library of nucleic acid molecules of claim 29, wherein the nucleic acid molecules are double-stranded.

31. A library of test constructs, wherein said library comprises at least 1×10⁶ test constructs, each of which comprises a different nucleic acid molecule positioned upstream of a reporter sequence, wherein each nucleic acid molecule comprises a stochastic sequence of at least 50 nucleotides, optionally wherein each nucleic acid molecule also comprises an identical pre-determined and fixed sequence either adjacent or within the stochastic sequence, wherein said pre-determined and fixed sequence does not include a promoter sequence itself capable of initiating transcription of a gene, and wherein each expression vector does not comprise a further separate sequence upstream of the reporter sequence that can modulate expression of the reporter sequence.

32. A library of host cells produced by introducing test constructs from the test construct library into host cells as defined in claim 1 or 2, wherein each host cell from the host cell library comprises a test construct as defined in any one of claims 13 to 17 or 23-25.
